SEQUENCE LISTING (4 sequences)

SEQ ID NO: 1 - DNA, 34 bp, Human immunodeficiency virus type 1
aattttcccattagtcctattgaaactgtaccag

SEQ ID NO: 2 - DNA, 27 bp, Human immunodeficiency virus type 1
ccccactaacttctgtatgtcattgac

SEQ ID NO: 3 - DNA, 24 bp, Human immunodeficiency virus type 1
gccgatagacaaggaactgtatcc

SEQ ID NO: 4 - DNA, 18 bp, Human immunodeficiency virus type 1
tgaaaaatatgcatcacc

(19) United States
(12) Patent Application Publication
(10) Pub. No.: US 2008/0241289 A1
(43) Pub. Date: Oct. 2, 2008

(54) PREPARATION AND UTILITY OF NON-NUCLEOSIDE REVERSE TRANSCRIPTASE INHIBITORS

(75) Inventors: Thomas G. Gant, Carlsbad, CA (US); Sepehr Sarshar, Cardiff by the Sea, CA (US)

Correspondence Address: GLOBAL PATENT GROUP - APX;Ms. LaVern Hall, 10411 Clayton Road, Suite 304, ST. LOUIS, MO, 63131, US

(73) Assignee: AUSPEX PHARMACEUTICALS, INC., Vista, CA (US)

(21) Appl. No.: 12/035,763

(22) Filed: Feb. 22, 2008

Related U.S. Application Data
(60) Provisional application No. 60/891,436, filed on Feb. 23, 2007.

(51) Int. Cl. A61K 36/752; A61K 31/551; C07D 487/14

(52) U.S. Cl. 424/736; 540/495; 514/220

(57) ABSTRACT

Disclosed herein are non-nucleoside reverse transcriptase inhibitors having structural Formula I, processes of preparation thereof, pharmaceutical compositions thereof, and the methods of their use thereof.

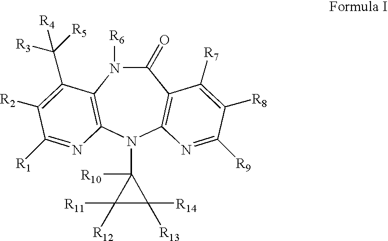

This application claims the benefit of priority of U.S. provisional application No. 60/891,436, filed Feb. 23, 2007 the disclosure of which is hereby incorporated by reference as if written herein in their entirety.

### FIELD

The present invention is directed non-nucleoside reverse transcriptase inhibitors and pharmaceutically acceptable salts and prodrugs thereof, the chemical synthesis thereof, and the medical use of such compounds for the treatment and/or management of infectious disorders.

### BACKGROUND

Nevirapine (“NVP”, Viramune®, Nevimune, Nevirex) is a purported inhibitor of HIV-1 reverse transcriptase. As such, it belongs to a class of drugs that is commonly divided into those agents that resemble naturally-occurring nucleosides, and those that do not. Nucleoside & Nucleotide analogs of reverse-transcriptase inhibitors (“NRTIs”) include abacavir (“ABC”), didanosine (“ddI”), emitracibine (“FTC”), lamivudine (“3TC”), stavudine (“d4T”), tenofovir (“TDF”), zidovudine (“AZT”), apricitabine, stampidine, elvucitabine, racivir and zalcitabine. The non-nucleoside reverse-transcriptase inhibitors (“NNRTIs”) include efavirenz (“EFV”), avirenz, etravirine, rilpivirine, loviride, delavirdine and nevirapine. These agents are typically administered in various combinations of NRTIs, NNRTIs, and other anti-HIV agents from the classes of protease inhibitors (“PIs”) and more recently, fusion inhibitors such as enfuvirtide (“Fuzeon”, T-20″), PRO 140, vicriviroc, and maraviroc.

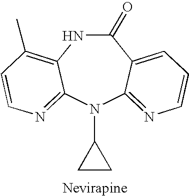

The benefits and shortcomings of this drug have been reviewed. Major shortcomings may be traced to metabolism-related phenomena. Metabolic studies have revealed that the methyl group is a primary site of oxidative metabolism. The resulting hydroxymethyl metabolite is believed to be further transformed to a toxic metabolite. This toxic metabolite is believed to be primarily responsible for the adverse events associated with nevirapine use. Common side effects include hepatotoxicity and allergic reactions. Stevens-Johnson syndrome, a potentially life threatening condition, is believed to be caused by a severe allergic reaction to niverapine or its metabolites. The hepatotoxicity of niverapine is roughly double that of efavirenz, another NNRTI. As a result, efavirenz is prescribed instead of niverapine for most conditions. Efavirenz, however, has been linked to birth defects in pregnant women and has been shown to cause central nervous system damage in some patients. Therefore, an improved NNRTI that significantly reduces the hepatoxicity and allergic reactions of nevirapine, while allowing the treatment of pregnant women (thus reducing the likelihood of mother-to-child transmission of HIV) would constitute a significant advance in anti-HIV therapy.

### SUMMARY OF THE INVENTION

Disclosed herein is a compound having structural Formula I:

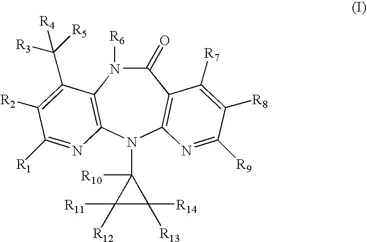

or a pharmaceutically acceptable salt, solvate, or prodrug thereof, wherein:
R₁, R₂, R₃, R₄, R₅, R₆, R₇, R₈, R₉, R₁₀, R₁₁, R₁₂, R₁₃, and R₁₄ are independently selected from the group consisting of hydrogen and deuterium;
at least one of R₁, R₂, R₃, R₄, R₅, R₆, R₇, R₈, R₉, R₁₀, R₁₁, R₁₂, R₁₃, and R₁₄ is deuterium; and
when R₃, R₄, and R₅ are deuterium then at least one of R₁, R₂, R₆, R₇, R₈, R₉, R₁₀, R₁₁, R₁₂, R₁₃, and R₁₄ is deuterium.

Also disclosed herein are pharmaceutical compositions comprising at least one compound as disclosed herein or a pharmaceutically acceptable salt, solvate, or prodrug thereof; in combination with one or more pharmaceutically acceptable excipients or carriers.

Further, disclosed herein are methods of inhibiting the activity of viral reverse transcriptase.

In addition, disclosed herein are methods of treating a subject having, suspected of having, or being prone to an infectious disorder, such as HIV.

Further, disclosed herein is a method for treating, preventing, or ameliorating an infectious disorder, which comprises administering to a subject a therapeutically effective amount of at least one compound as disclosed herein or a pharmaceutically acceptable salt, solvate, or prodrug thereof.

Also disclosed herein are articles of manufacture and kits containing compounds as disclosed herein. By way of example only a kit or article of manufacture can include a container (such as a bottle) with a desired amount of at least one compound (or pharmaceutical composition of a compound) as disclosed herein. Further, such a kit or article of manufacture can further include instructions for using said compound (or pharmaceutical composition of a compound) as disclosed herein. The instructions can be attached to the container, or can be included in a package (such as a box or a plastic or foil bag) holding the container.

In another aspect is the use of at least one compound as disclosed herein in the manufacture of a medicament for treating a disorder in a subject in which viruses contribute to the pathology and/or symptomology of the disorder. In a further or alternative embodiment, said disorder is an infectious disorder.

In another aspect are processes for preparing a compound as disclosed herein or other pharmaceutically acceptable derivative thereof such as a salt, solvate, or prodrug, as an antiinfective agent.

Also disclosed herein are processes for formulating pharmaceutical compositions with a compound disclosed herein.

In certain embodiments said pharmaceutical composition is suitable for oral, parenteral, or intravenous infusion administration.

In other embodiments said pharmaceutical composition comprises a suspension.

In yet other embodiments said pharmaceutical composition comprises a tablet, or capsule.

In certain embodiments the compounds as disclosed herein are administered in a dose of 0.5 milligrams to 1000 milligrams.

In yet further embodiments said pharmaceutical compositions further comprise another therapeutic agent.

In other embodiments said therapeutic agent is selected from the group consisting of: NRTIs, NNRTIs, protease inhibitors, entry or fusin inhibitors, integrase inhibitors, maturation inhibitors, antiviral associated agents, HIV fixed drug combinations, antifungal agents, antibacterials, antimycobacterial agents, sepsis treatments, steroidal drugs, anticoagulants, thrombolytics, non-steroidal anti-inflammatory agents, antiplatelet agents, endothelin converting enzyme (ECE) inhibitors, thromboxane receptor antagonists, potassium channel openers, thrombin inhibitors, growth factor inhibitors, platelet activating factor (PAF) antagonists, anti-platelet agents, Factor VIIa Inhibitors, Factor Xa Inhibitors, renin inhibitors, neutral endopeptidase (NEP) inhibitors, vasopepsidase inhibitors, HMG CoA reductase inhibitors, squalene synthetase inhibitors, fibrates, bile acid sequestrants, anti-atherosclerotic agents, MTP Inhibitors, calcium channel blockers, potassium channel activators, alpha-adrenergic agents, beta-adrenergic agents, antiarrhythmic agents, diuretics, anti-diabetic agents, PPAR-gamma agonists, mineralocorticoid receptor antagonists, aP2 inhibitors, phosphodiesterase inhibitors, protein tyrosine kinase inhibitors, antiinflammatories, antiproliferatives, chemotherapeutic agents, immunosuppressants, anticancer agents, cytotoxic agents, antimetabolites, farnesyl-protein transferase inhibitors, hormonal agents, microtubule-disruptor agents, microtubule-stablizing agents, topoisomerase inhibitors, prenyl-protein transferase inhibitors, cyclosporins, TNF-alpha inhibitors, cyclooxygenase-2 (COX-2) inhibitors, gold compounds, and platinum coordination complexes.

In yet further embodiments said therapeutic agent(s) is/are one or more nucleoside NRTIs.

In certain embodiments said NRTI is selected from the group consisting of abacavir, didanosine, emtricitabine, lamivudine, stavudine, zidovudine, tenofovir, apricitabine, stampidine, elvucitabine, racivir and zalcitabine.

In other embodiments the NRTI is zidovudine.

In further embodiments the NRTIs are zidovudine and lamivudine.

In certain embodiments the NRTI is tenofovir.

In yet other embodiments the NRTIs are tenofovir and emitricitabine.

In further embodiments the NRTI is stavudine.

In further embodiments the NRTI are stavudine and lamivudine.

In other embodiments said therapeutic agent is a NNRTI.

In certain embodiments said NNRTI is selected from the group consisting of avirenz, nevirapine, etravirine, rilpivirine, loviride and delavirdine.

In yet further embodiments said therapeutic agent is a protease inhibitor.

In certain embodiments said protease inhibitor is selected from the group consisting of atazanavir, darunavir, fosamprenavir, lopinavir, nelfinavir, ritonavir, saquinavir, tipranavir, amprenavir and indinavir.

In other embodiments said therapeutic agent is an entry or fusion inhibitor.

In certain embodiments said entry or fusion inhibitor is selected from the group consisting of enfuvirtide, maraviroc, PRO140 and vicriviroc.

In yet further embodiments said therapeutic agent is an integrase inhibitor.

In certain embodiments said integrase inhibitor is selected from the group consisting of raltegravir, and elvitegravir.

In other embodiments said therapeutic agent is a maturation inhibitor.

In certain embodiments said maturation inhibitor is selected from the group consisting of bevirimat and vivecon.

In yet further embodiments said therapeutic agent is an antiviral associated agent.

In certain embodiments said antiviral associated agent is selected from the group consisting of foscarnet, chloroquine, quinoline, grapefruit juice, hydroxyurea, leflunomide, mycophenolic acid, resveratrol, ritonavir, epigallocatechin gallate, portmanteau inhibitors, Globoidnan A, griffithsin, diarylpyrimidines, and Calanolide A.

In other embodiments said therapeutic agent is a HIV fixed drug combination.

In yet further embodiments said HIV fixed drug combination is selected from the group consisting of Combivir®, Atripla®, Trizivir®, Truvada®, Kaletra®, and Epzicom®.

In certain embodiments of the present invention a method of treating a subject suffering from an infectious disorder comprises administering to said subject a therapeutically effective amount of a compound of a compound having structural Formula II:

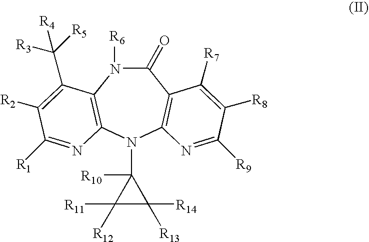

or a pharmaceutically acceptable salt, solvate, or prodrug thereof, wherein:

R₁, R₂, R₃, R₄, R₅, R₆, R₇, R₈, R₉, R₁₀, R₁₁, R₁₂, R₁₃, and R₁₄ are independently selected from the group consisting of hydrogen and deuterium; and

at least one of R₁, R₂, R₃, R₄, R₅, R₆, R₇, R₈, R₉, R₁₀, R₁₁, R₁₂, R₁₃, and R₁₄ is deuterium.

In certain further embodiments said infectious disorder is cause by a virus.

In other embodiments said virus is a retrovirus.

In yet further embodiments said retrovirus is selected from the group consisting of alpharetrovirus, betaretrovirus, gammaretrovirus, deltaretrovirus, epsilonretrovirus, lentivirus, spumavirus, and endogenous retrovirus.

In certain embodiments said retrovirus is a lentivirus.

In other embodiments said lentivirus is HIV type 1.

In yet further embodiments said infectious disorder can be ameliorated by administering an antiinfective agent.

In other embodiments said compound has at least one of the following properties:
a) decreased inter-individual variation in plasma levels of said compound or a metabolite thereof as compared to the non-isotopically enriched compound;
b) increased average plasma levels of said compound per dosage unit thereof as compared to the non-isotopically enriched compound;
c) decreased average plasma levels of at least one metabolite of said compound per dosage unit thereof as compared to the non-isotopically enriched compound;
d) increased average plasma levels of at least one metabolite of said compound per dosage unit thereof as compared to the non-isotopically enriched compound; and
e) an improved clinical effect during the treatment in said subject per dosage unit thereof as compared to the non-isotopically enriched compound.

In yet further embodiments said compound has at least two of the following properties:
a) decreased inter-individual variation in plasma levels of said compound or a metabolite thereof as compared to the non-isotopically enriched compound;
b) increased average plasma levels of said compound per dosage unit thereof as compared to the non-isotopically enriched compound;
c) decreased average plasma levels of at least one metabolite of said compound per dosage unit thereof as compared to the non-isotopically enriched compound;
d) increased average plasma levels of at least one metabolite of said compound per dosage unit thereof as compared to the non-isotopically enriched compound; and
e) an improved clinical effect during the treatment in said subject per dosage unit thereof as compared to the non-isotopically enriched compound.

In certain embodiments said compound has a decreased metabolism by at least one polymorphically-expressed cytochrome P₄₅₀ isoform in said subject per dosage unit thereof as compared to the non-isotopically enriched compound.

In other embodiments said cytochrome P₄₅₀ isoform is selected from the group consisting of CYP2C8, CYP2C9, CYP2C19, and CYP2D6.

In yet further embodiments said compound is characterized by decreased inhibition of at least one cytochrome P₄₅₀ or monoamine oxidase isoform in said subject per dosage unit thereof as compared to the non-isotopically enriched compound.

In certain embodiments said cytochrome P₄₅₀ or monoamine oxidase isoform is selected from the group consisting of CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4 A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, CYP51, MAO_{A}, and MAO_{B}.

In other embodiments said compound reduces or eliminates a deleterious change in a diagnostic hepatobiliary function endpoint, as compared to the corresponding non-isotopically enriched compound.

In other embodiments said diagnostic hepatobiliary function endpoint is selected from the group consisting of alanine aminotransferase (“ALT”), serum glutamic-pyruvic transaminase (“SGPT”), aspartate aminotransferase (“AST,” “SGOT”), ALT/AST ratios, serum aldolase, alkaline phosphatase (“ALP”), ammonia levels, bilirubin, gamma-glutamyl transpeptidase (“GGTP,” “γ-GTP,” “GGT”), leucine aminopeptidase (“LAP”), liver biopsy, liver ultrasonography, liver nuclear scan, 5′-nucleotidase, and blood protein.

### INCORPORATION BY REFERENCE

All publications and references cited herein, including those in the background section, are expressly incorporated herein by reference in their entirety. However, with respect to any similar or identical terms found in both the incorporated publications or references and those explicitly put forth or defined in this document, then those terms definitions or meanings explicitly put forth in this document shall control in all respects.

### DETAILED DESCRIPTION

To facilitate understanding of the disclosure set forth herein, a number of terms are defined below. Generally, the nomenclature used herein and the laboratory procedures in organic chemistry, medicinal chemistry, and pharmacology described herein are those well known and commonly employed in the art. Unless defined otherwise, all technical and scientific terms used herein generally have the same meaning as commonly understood in the art to which this disclosure belongs. In the event that there is a plurality of definitions for a term used herein, those in this section prevail unless stated otherwise.

As used herein, the singular forms “a,” “an,” and “the” may refer to plural articles unless specifically stated otherwise.

The term “subject” refers to an animal, including, but not limited to, a primate (e.g., human monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein, for example, to a mammalian subject, such as a human patient.

The terms “treat,” “treating,” and “treatment” are meant to include alleviating or abrogating a disorder; or one or more of the symptoms associated with the disorder; or alleviating or eradicating the cause(s) of the disorder itself.

The terms “prevent,” “preventing,” and “prevention” refer to a method of delaying or precluding the onset of a disorder; and/or its attendant symptoms, barring a subject from acquiring a disorder or reducing a subject's risk of acquiring a disorder.

The term “therapeutically effective amount” refers to the amount of a compound that, when administered, is sufficient to prevent development of, or alleviate to some extent, one or more of the symptoms of the disorder being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenecity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156%, deuterium enrichment at any position in a compound synthesized using non-enriched starting materials is about 0.0156%. The deuterium enrichment can be determined using conventional analytical methods known to one of ordinary skill in the art, including mass spectrometry and nuclear magnetic resonance spectroscopy.

The term “is/are deuterium,” when used to describe a given position in a molecule such as R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₃, R₂₄, R₂₅, R₂₆, R₂₇, and R₂₈, or the symbol “D,” when used to represent a given position in a drawing of a molecular structure, means that the specified position is enriched with deuterium above the naturally occurring distribution of deuterium. In an embodiment deuterium enrichment is of no less than about 1%, in another no less than about 5%, in another no less than about 10%, in another no less than about 20%, in another no less than about 50%, in another no less than about 70%, in another no less than about 80%, in another no less than about 90%, in another no less than about 95%, or in another no less than about 98% of deuterium at the specified position.

The term “isotopic enrichment” refers to the percentage of incorporation of a less prevalent isotope of an element at a given position in a molecule in the place of the more prevalent isotope of the element.

The term “non-isotopically enriched” refers to a molecule in which the percentages of the various isotopes are substantially the same as the naturally occurring percentages.

The terms “substantially pure” and “substantially homogeneous” mean sufficiently homogeneous to appear free of readily detectable impurities as determined by standard analytical methods used by one of ordinary skill in the art, including, but not limited to, thin layer chromatography (TLC), gel electrophoresis, high performance liquid chromatography (HPLC), infrared spectroscopy (IR), gas chromatography (GC), Ultraviolet Spectroscopy (UV), nuclear magnetic resonance (NMR), atomic force spectroscopy, and mass spectroscopy (MS); or sufficiently pure such that further purification would not detectably alter the physical and chemical properties, or biological and pharmacological properties, such as enzymatic and biological activities, of the substance. In certain embodiments, “substantially pure” or “substantially homogeneous” refers to a collection of molecules, wherein at least about 50%, at least about 70%, at least about 80%, at least about 90%, at least about 95%, at least about 98%, at least about 99%, or at least about 99.5% of the molecules are a single compound, including a racemic mixture or single stereoisomer thereof, as determined by standard analytical methods.

The term “about” or “approximately” means an acceptable error for a particular value as determined by one of ordinary skill in the art, which depends in part on how the value is measured or determined. In certain embodiments, “about” can mean 1 or more standard deviations.

The terms “active ingredient” and “active substance” refer to a compound, which is administered, alone or in combination with one or more pharmaceutically acceptable excipients or carriers, to a subject for treating, preventing, or ameliorating one or more symptoms of a disorder.

The terms “drug,” “therapeutic agent,” and “chemotherapeutic agent” refer to a compound, or a pharmaceutical composition thereof, which is administered to a subject for treating, preventing, or ameliorating one or more symptoms of a disorder.

The term “disorder” as used herein is intended to be generally synonymous, and is used interchangeably with, the terms “disease,” “syndrome” and “condition” (as in medical condition), in that all reflect an abnormal condition of the body or of one of its parts that impairs normal functioning and is typically manifested by distinguishing signs and symptoms.

The term “release controlling excipient” refers to an excipient whose primary function is to modify the duration or place of release of the active substance from a dosage form as compared with a conventional immediate release dosage form.

The term “nonrelease controlling excipient” refers to an excipient whose primary function do not include modifying the duration or place of release of the active substance from a dosage form as compared with a conventional immediate release dosage form.

The term “infectious disorder” refers to a disorder caused by an infection, a suspected infection, an anticipated infection, or an exposure to an infectious agent. An infectious disorder includes a viral-mediated disorder.

The term “antiinfective agent” refers any substance that ameliorates, lessens, obliterates, removes, kills or prevents the spreading of infectious agents or organisms in order to prevent the spread of infection. Antiinfective agents are used to treat disorders caused by bacteria, viruses, protozoa, worms, fungi, and yeast. An antiifective agent can be used to treat and infectious disorder.

The term “viral-mediated disorder” as used herein refers to a disorder that is characterized by a viral infection, and when the viral activity is antagonized, inhibited, or eliminated, leads to the amelioration of other abnormal biological processes. A viral-mediated disorder may be completely or partially mediated by administering an antiinfective agent. In particular, a viral-mediated disorder is one in which modulation of viral activity results in some effect on the underlying disorder, for example, administering an antiinfective agent results in some improvement in at least some of the patients being treated.

The term “reverse transcriptase” or “RNA-dependent DNA polymerase” refers to a DNA polymerase enzyme that transcribes single-stranded RNA into single-stranded DNA. Normal transcription involves the synthesis of RNA from DNA; hence, reverse transcription is the reverse of normal transcription. Reverse transcriptases are ubiquitous to retroviruses. Common examples include HIV-1 reverse transcriptase, M-MLV reverse transcriptase from the Moloney murine leukemia virus, AMV reverse transcriptase from the avian myeloblastosis virus, among others.

The term “nucleoside and nucleotide reverse transcriptase inhibitors” or “NRTI” refers to nucleosides, nucleotides, and analogues thereof which mimic natural nucleoside and nucleotide bases. When a retrovirus, such as HIV type 1, replicates its viral RNA using reverse transcriptase, these mimics compete with natural nucleoside and nucleotide base pairs for DNA elongation. Incorporation of one of these mimics results in DNA chain termination.

The term “non-nucleoside reverse transcriptase inhibitors” or “NNRTI” refers to compounds which bind to a retrovirus reverse transcriptase, such as HIV type 1's reverse transcriptase, and inhibits its enzymatic activity. The binding by the “NNRTI” causes a conformational shift in the reverse transcriptase which prevents the enzyme from binding nucleoside and nucleotide bases, resulting in DNA chain termination.

The term “protease inhibitor” or “PI” refers to compounds which bind to active site of a retroviral protease enzyme, such as HIV's protease enzyme. The binding by the “PI” causes a conformational shift in the retroviral protease enzyme, making it no longer able to cleave large viral precursor proteins into smaller functional proteins. Viruses that are produced are defective and unable to infect other cells.

The term “entry/fusion inhibitor” or “entry or fusion inhibitor” refers to compounds which interfere with the binding, fusion and entry of an HIV virion to a human cell. By blocking this step in HIV's replication cycle, such agents slow the progression from HIV infection to AIDS.

The term “integrase inhibitor” refers to compounds which interfere with the action of integrase, an enzyme that integrates genetic material from the virus into the host's DNA. Integrase inhibitors are also called strand transfer inhibitors. Strand transfer refers to the process by which the viral DNA strands are transferred from the viral genome to the host genome. Integrase inhibitors may be taken in combination with other types of retroviral drugs to minimize adaptation by the virus.

The term “maturation inhibitor” refers to compounds which interfere with the assembly and budding of virion particles, by binding to the viral gag polyprotein. The bound gag polyprotein can no longer be processed to functional subunits by viral protease enzymes. The resulting virus particles are structurally defective and are incapable of spreading infection.

The term “antiviral associated agent” refers to compounds which have been shown to enhance the effectiveness of antiviral drugs or have antiviral properties.

The term “HIV fixed drug combination” refers to multiple antiretroviral drugs combined into a single pill, which helps reduce pill burden. The formulations may combine different classes of antiretrovirals or contain only a single class. Licensed fixed dose combinations include, but are not limited to; GlaxoSmithKline's Combivir® (zidovudine and lamivudine), Trizivir® (abacavir, zidovudine, and lamivudine), and Epzicom® or Kivexa® (abacavir and lamivudine); Abbott Laboratories's Kaletra® (lopinavir and ritonavir); Gilead Sciences's Truvada® (emtricitabine and tenofovir); and Gilead Sciences/Bristol-Myers Squibb's Atripla® (efavirenz, emtricitabine, and tenofovir).

The term “protecting group” or “removable protecting group” refers to a group which, when bound to a functionality, such as the oxygen atom of a hydroxyl or carboxyl group, or the nitrogen atom of an amino group, prevents reactions from occurring at that functional group, and which can be removed by a conventional chemical or enzymatic step to reestablish the functional group (Greene and Wuts, Protective Groups in Organic Synthesis, 3^{rd} Ed., John Wiley & Sons, New York, N.Y., 1999).

The term “halogen”, “halide” or “halo” includes fluorine, chlorine, bromine, and iodine.

The term “leaving group” (LG) refers to any atom (or group of atoms) that is stable in its anion or neutral form after it has been displaced by a nucleophile and as such would be obvious to one of ordinary skill and knowledge in the art. The definition of “leaving group” includes but is not limited to: water, methanol, ethanol, chloride, bromide, iodide, an alkylsulfonate, for example methanesulfonate, ethanesulfonate and the like, an arylsulfonate, for example benzenesulfonate, tolylsulfonate and the like, a perhaloalkanesulfonate, for example trifluoromethanesulfonate, trichloromethanesulfonate and the like, an alkylcarboxylate, for example acetate and the like, a perhaloalkylcarboxylate, for example trifluoroacetate, trichloroacetate and the like, an arylcarboxylate, for example benzoate and the like.

The term “coupling reagent” refers to any reagent that will activate the carbonyl of a carboxylic acid and facilitate the formation of an ester or amide bond. The definition of “coupling reagent” includes but is not limited to: cuprous iodide, acetyl chloride, ethyl chloroformate, dicyclohexylcarbodiimide (DCC), diisopropyl carbodiimide (DIC), 1-ethyl-3-(3-dimethylaminopropyl) carbodiimide (EDCI), N-hydroxybenzotriazole (HOBT), N-hydroxysuccinimide (HOSu), 4-nitrophenol, pentafluorophenol, 2-(1H-benzotriazole-1-yl)-1,1,3,3-tetramethyluronium tetrafluoroborate (TBTU), O-benzotriazole-N,N,N′N′-tetramethyluronium hexafluorophosphate (HBTU), benzotriazole-1-yl-oxy-tris-(dimethylamino)-phosphonium hexafluorophosphate (BOP), benzotriazole-1-yl-oxy-tris-pyrrolidinophosphonium hexafluorophosphate, bromo-trispyrrolidino-phosphonium hexafluorophosphate, 2-(5-norbornene-2,3-dicarboximido)-1,1,3,3-tetramethyluronium tetrafluoroborate (TNTU), O—(N-succinimidyl)-1,1,3,3-tetramethyluronium tetrafluoroborate (TSTU), tetramethylfluoroformamidinium hexafluorophosphate and the like.

The terms “alkyl” and “substituted alkyl” are interchangeable and include substituted, optionally substituted and unsubstituted C₁-C₁₀ straight chain saturated aliphatic hydrocarbon groups, substituted, optionally substituted and unsubstituted C₂-C₁₀ straight chain unsaturated aliphatic hydrocarbon groups, substituted, optionally substituted and unsubstituted C₂-C₁₀ branched saturated aliphatic hydrocarbon groups, substituted and unsubstituted C₂-C₁₀ branched unsaturated aliphatic hydrocarbon groups, substituted, optionally substituted and unsubstituted C₃-C₈ cyclic saturated aliphatic hydrocarbon groups, substituted, optionally substituted and unsubstituted C₅-C₈ cyclic unsaturated aliphatic hydrocarbon groups having the specified number of carbon atoms. For example, the definition of “alkyl” shall include but is not limited to: methyl (Me), trideuteromethyl (-CD₃), ethyl (Et), propyl (Pr), butyl (Bu), pentyl, hexyl, heptyl, octyl, nonyl, decyl, undecyl, ethenyl, propenyl, butenyl, penentyl, hexenyl, heptenyl, octenyl, nonenyl, decenyl, undecenyl, isopropyl (i-Pr), isobutyl (i-Bu), tert-butyl (t-Bu), sec-butyl (s-Bu), isopentyl, neopentyl, cyclopropyl, cyclobutyl, cyclopentyl, cyclohexyl, cycloheptyl, cyclooctyl, cyclopentenyl, cyclohexenyl, cycloheptenyl, cyclooctenyl, methylcyclopropyl, ethylcyclohexenyl, butenylcyclopentyl, adamantyl, norbornyl and the like. Alkyl substituents are independently selected from the group consisting of hydrogen, deuterium, halogen, —OH, —SH, —NH₂, —CN, —NO₂, ═O, ═CH₂, trihalomethyl, carbamoyl, arylC₀₋₁₀alkyl, heteroarylC₀₋₁₀alkyl, C₁₋₁₀alkyloxy, arylC₀₋₁₀alkyloxy, C₁₋₁₀alkylthio, arylC₀₋₁₀alkylthio, C₁₋₁₀alkylamino, arylC₀₋₁₀alkylamino, N-aryl-N—C₀₋₁₀alkylamino, C₁₋₁₀alkylcarbonyl, arylC₀₋₁₀alkylcarbonyl, C₁₋₁₀alkylcarboxy, arylC₀₋₁₀alkylcarboxy, C₁₋₁₀alkylcarbonylamino, arylC₀₋₁₀alkylcarbonylamino, tetrahydrofuryl, morpholinyl, piperazinyl, hydroxypyronyl, —C₀₋₁₀alkylCOOR₃₀ and —C₀₋₁₀alkylCONR₃₁R₃₂ wherein R₃₀, R₃₁ and R₃₂ are independently selected from the group consisting of hydrogen, deuterium, alkyl, aryl, or R₃₂ and R₃₃ are taken together with the nitrogen to which they are attached forming a saturated cyclic or unsaturated cyclic system containing 3 to 8 carbon atoms with at least one substituent as defined herein.

The term “aryl” represents an unsubstituted, mono-, or polysubstituted monocyclic, polycyclic, biaryl aromatic groups covalently attached at any ring position capable of forming a stable covalent bond, certain preferred points of attachment being apparent to those skilled in the art (e.g., 3-phenyl, 4-naphthyl and the like). The aryl substituents are independently selected from the group consisting of hydrogen, deuterium, halogen, —OH, —SH, —CN, —NO₂, trihalomethyl, hydroxypyronyl, C₁₋₁₀alkyl, arylC₀₋₁₀alkyl, C₀₋₁₀alkyloxyC₀₋₁₀alkyl, arylC₀₋₁₀alkyloxyC₀₋₁₀alkyl, C₀₋₁₀alkylthioC₀₋₁₀alkyl, arylC₀₋₁₀alkylthioC₀₋₁₀alkyl, C₀₋₁₀alkylaminoC₀₋₁₀alkyl, arylC₀₋₁₀alkylaminoC₀₋₁₀alkyl, N-aryl-N—C₀₋₁₀alkylaminoC₀₋₁₀alkyl, C₁₋₁₀alkylcarbonylC₀₋₁₀alkyl, arylC₀₋₁₀alkylcarbonylC₀₋₁₀alkyl, C₁₋₁₀alkylcarboxyC₀₋₁₀alkyl, arylC₀₋₁₀alkylcarboxyC₀₋₁₀alkyl, C₁₋₁₀alkylcarbonylaminoC₀₋₁₀alkyl, arylC₀₋₁₀alkylcarbonylaminoC₀₋₁₀alkyl, —C₀₋₁₀alkylCOOR₃₀, and —C₀₋₁₀alkylCONR₃₁R₃₂ wherein R₃₀, R₃₁ and R₃₂ are independently selected from the group consisting of hydrogen, deuterium, alkyl, aryl or R₃₁ and R₃₂ are taken together with the nitrogen to which they are attached forming a saturated cyclic or unsaturated cyclic system containing 3 to 8 carbon atoms with at least one substituent as defined above.

The definition of “aryl” includes but is not limited to phenyl, pentadeuterophenyl, biphenyl, naphthyl, dihydronaphthyl, tetrahydronaphthyl, indenyl, indanyl, azulenyl, anthryl, phenanthryl, fluorenyl, pyrenyl and the like.

In light of the purposes described in the present disclosure, all references to “alkyl” and “aryl” groups or any groups ordinarily containing C—H bonds may include partially or fully deuterated versions as required to affect the improvements outlined herein.

### Deuterium Kinetic Isotope Effect

In an attempt to eliminate foreign substances, such as therapeutic agents, from its circulation system, the animal body expresses various enzymes, such as the cytochrome P₄₅₀ enzymes or CYPs, esterases, proteases, reductases, dehydrogenases, and monoamine oxidases, to react with and convert these foreign substances to more polar intermediates or metabolites for renal excretion. Some of the most common metabolic reactions of pharmaceutical compounds involve the oxidation of a carbon-hydrogen (C—H) bond to either a carbon-oxygen (C—O) or carbon-carbon (C—C) π-bond. The resultant metabolites may be stable or unstable under physiological conditions, and can have substantially different pharmacokinetic, pharmacodynamic, and acute and long-term toxicity profiles relative to the parent compounds. For most drugs, such oxidations are generally rapid and ultimately lead to administration of multiple or high daily doses.

The relationship between the activation energy and the rate of reaction may be quantified by the Arrhenius equation, k=Ae^{-Eact/RT}, where E_{act} is the activation energy, T is temperature, R is the molar gas constant, k is the rate constant for the reaction, and A (the frequency factor) is a constant specific to each reaction that depends on the probability that the molecules will collide with the correct orientation. The Arrhenius equation states that the fraction of molecules that have enough energy to overcome an energy barrier, that is, those with energy at least equal to the activation energy, depends exponentially on the ratio of the activation energy to thermal energy (RT), the average amount of thermal energy that molecules possess at a certain temperature.

The transition state in a reaction is a short lived state (on the order of 10⁻¹⁴ sec) along the reaction pathway during which the original bonds have stretched to their limit. By definition, the activation energy E_{act} for a reaction is the energy required to reach the transition state of that reaction. Reactions that involve multiple steps will necessarily have a number of transition states, and in these instances, the activation energy for the reaction is equal to the energy difference between the reactants and the most unstable transition state. Once the transition state is reached, the molecules can either revert, thus reforming the original reactants, or new bonds form giving rise to the products. This dichotomy is possible because both pathways, forward and reverse, result in the release of energy. A catalyst facilitates a reaction process by lowering the activation energy leading to a transition state. Enzymes are examples of biological catalysts that reduce the energy necessary to achieve a particular transition state.

A carbon-hydrogen bond is by nature a covalent chemical bond. Such a bond forms when two atoms of similar electronegativity share some of their valence electrons, thereby creating a force that holds the atoms together. This force or bond strength can be quantified and is expressed in units of energy, and as such, covalent bonds between various atoms can be classified according to how much energy must be applied to the bond in order to break the bond or separate the two atoms.

The bond strength is directly proportional to the absolute value of the ground-state vibrational energy of the bond. This vibrational energy, which is also known as the zero-point vibrational energy, depends on the mass of the atoms that form the bond. The absolute value of the zero-point vibrational energy increases as the mass of one or both of the atoms making the bond increases. Since deuterium (D) has twice the mass of hydrogen (H), it follows that a C—D bond is stronger than the corresponding C—H bond. Compounds with C—D bonds are frequently indefinitely stable in H₂O, and have been widely used for isotopic studies. If a C—H bond is broken during a rate-determining step in a chemical reaction (i.e. the step with the highest transition state energy), then substituting a deuterium for that hydrogen will cause a decrease in the reaction rate and the process will slow down. This phenomenon is known as the Deuterium Kinetic Isotope Effect (DKIE). The magnitude of the DKIE can be expressed as the ratio between the rates of a given reaction in which a C—H bond is broken, and the same reaction where deuterium is substituted for hydrogen. The DKIE can range from about 1 (no isotope effect) to very large numbers, such as 50 or more, meaning that the reaction can be fifty, or more, times slower when deuterium is substituted for hydrogen. High DKIE values may be due in part to a phenomenon known as tunneling, which is a consequence of the uncertainty principle. Tunneling is ascribed to the small mass of a hydrogen atom, and occurs because transition states involving a proton can sometimes form in the absence of the required activation energy. Because deuterium has more mass than hydrogen, it statistically has a much lower probability of undergoing this phenomenon. Substitution of tritium for hydrogen results in yet a stronger bond than deuterium and gives numerically larger isotope effects

Discovered in 1932 by Urey, deuterium (D) is a stable and non-radioactive isotope of hydrogen. It was the first isotope to be separated from its element in pure form and has twice the mass of hydrogen, and makes up about 0.02% of the total mass of hydrogen (in this usage meaning all hydrogen isotopes) on earth. When two deuterium atoms bond with one oxygen, deuterium oxide (D₂O or “heavy water”) is formed. D₂O looks and tastes like H₂O, but has different physical properties. It boils at 101.41° C. and freezes at 3.79° C. Its heat capacity, heat of fusion, heat of vaporization, and entropy are all higher than H₂O. It is more viscous and has different solubilizng properties than H₂O.

When pure D₂O is given to rodents, it is readily absorbed and reaches an equilibrium level that is usually about eighty percent of the concentration of what was consumed. The quantity of deuterium required to induce toxicity is extremely high. When 0% to as much as 15% of the body water has been replaced by D₂O, animals are healthy but are unable to gain weight as fast as the control (untreated) group. When about 15% to about 20% of the body water has been replaced with D₂O, the animals become excitable. When about 20% to about 25% of the body water has been replaced with D₂O, the animals are so excitable that they go into frequent convulsions when stimulated. Skin lesions, ulcers on the paws and muzzles, and necrosis of the tails appear. The animals also become very aggressive; males becoming almost unmanageable. When about 30%, of the body water has been replaced with D₂O, the animals refuse to eat and become comatose. Their body weight drops sharply and their metabolic rates drop far below normal, with death occurring at about 30 to about 35% replacement with D₂O. The effects are reversible unless more than thirty percent of the previous body weight has been lost due to D₂O. Studies have also shown that the use of D₂O can delay the growth of cancer cells and enhance the cytotoxicity of certain antineoplastic agents.

Tritium (T) is a radioactive isotope of hydrogen, used in research, fusion reactors, neutron generators and radiopharmaceuticals. Mixing tritium with a phosphor provides a continuous light source, a technique that is commonly used in wristwatches, compasses, rifle sights and exit signs. It was discovered by Rutherford, Oliphant and Harteck in 1934, and is produced naturally in the upper atmosphere when cosmic rays react with H₂ molecules. Tritium is a hydrogen atom that has 2 neutrons in the nucleus and has an atomic weight close to 3. It occurs naturally in the environment in very low concentrations, most commonly found as T₂O, a colorless and odorless liquid. Tritium decays slowly (half-life=12.3 years) and emits a low energy beta particle that cannot penetrate the outer layer of human skin. Internal exposure is the main hazard associated with this isotope, yet it must be ingested in large amounts to pose a significant health risk. As compared with deuterium, a lesser amount of tritium must be consumed before it reaches a hazardous level.

Deuteration of pharmaceuticals to improve pharmacokinetics (PK), pharmacodynamics (PD), and toxicity profiles, has been demonstrated previously with some classes of drugs. For example, the DKIE was used to decrease the hepatotoxicity of halothane by presumably limiting the production of reactive species such as trifluoroacetyl chloride. However, this method may not be applicable to all drug classes. For example, deuterium incorporation can lead to metabolic switching. The concept of metabolic switching asserts that xenogens, when sequestered by Phase I enzymes, may bind transiently and re-bind in a variety of conformations prior to the chemical reaction (e.g., oxidation). This hypothesis is supported by the relatively vast size of binding pockets in many Phase I enzymes and the promiscuous nature of many metabolic reactions. Metabolic switching can potentially lead to different proportions of known metabolites as well as altogether new metabolites. This new metabolic profile may impart more or less toxicity. Such pitfalls are non-obvious and are not predictable a priori for any drug class.

### Deuterated Non-Nucleoside Reverse Transcriptase Inhibitor Derivatives

Nevirapine (Viramune®) is a non-nucleoside reverse transcriptase inhibitor. The carbon-hydrogen bonds of nevirapine contain a naturally occurring distribution of hydrogen isotopes, namely ¹H or protium (about 99.9844%), ²H or deuterium (about 0.0156%), and ³H or tritium (in the range between about 0.5 and 67 tritium atoms per 10¹⁸ protium atoms). Increased levels of deuterium incorporation produce a detectable Kinetic Isotope Effect (KIE) that could affect the pharmacokinetic, pharmacologic and/or toxicologic parameters of such antiinfective agents relative to compounds having naturally occurring levels of deuterium.

Based on discoveries made in our laboratory, as well as considering the KIE literature, nvirapine is likely metabolized in humans at the methyl C—H bonds. The oxidative metabolism of these methyl C—H bonds gives rise to several metabolites, including a hydroxymethyl metabolite. The hydroxymethyl metabolite is biotransformed into a sulfated metabolite which is then converted further to a reactive species by sulfotransferases. This reactive species accounts for most if not all of niverapine's toxicity. While the toxicity and pharmacology of the resultant metabolite/s are not known with absolute certainty, limiting the production of such reactive and toxic metabolites has the potential to decrease the danger of the administration of such drugs, and may even allow increased dosage and concomitant increased efficacy.

Aspects of the present invention disclosed herein describe a novel approach to designing and synthesizing new analogs of these antiinfective agents through chemical modifications and derivations of the carbon-hydrogen bonds of these antiinfective agents and/or of the chemical precursors used to synthesize said antiinfective agents. Suitable modifications of certain carbon-hydrogen bonds into carbon-deuterium bonds may generate novel antiinfective agents with unexpected and non-obvious improvements of pharmacological, pharmacokinetic and toxicological properties in comparison to the non-isotopically enriched antiinfective agents. The deuterated analogs of this invention have the potential to uniquely maintain the beneficial aspects of the non-isotopically enriched drugs while substantially increasing the maximum tolerated dose, decreasing toxicity, increasing the half-life (T_{1/2}), lowering the maximum plasma concentration (Cₘₐₓ) of the minimum efficacious dose (MED), lowering the efficacious dose and thus decreasing non-mechanism-related toxicity, and/or lowering the probability of drug-drug interactions. These analogs also have strong potential to reduce the cost-of-goods (COG) owing to the ready availability of inexpensive sources of deuterated reagents combined with previously mentioned potential for lowering the therapeutic dose.

Various deuteration patterns can be used to a) reduce or eliminate unwanted metabolites, b) increase the half-life of the parent drug, c) decrease the number of doses needed to achieve a desired effect, d) decrease the amount of a dose needed to achieve a desired effect, e) increase the formation of active metabolites, if any are formed, and/or f) decrease the production of deleterious metabolites in specific tissues and/or create a more effective drug and/or a safer drug for polypharmacy, whether the polypharmacy be intentional or not. The deuteration approach has strong potential to slow the metabolism via various oxidative mechanisms.

In one embodiment, disclosed herein is a compound having structural Formula I:

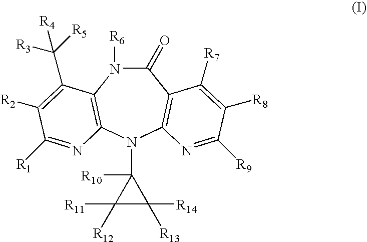

or a pharmaceutically acceptable salt, solvate, or prodrug thereof, wherein:

R₁, R₂, R₃, R₄, R₅, R₆, R₇, R₈, R₉, R₁₀, R₁₁, R₁₂, R₁₃, and R₁₄ are independently selected from the group consisting of hydrogen and deuterium;

at least one of R₁, R₂, R₃, R₄, R₅, R₆, R₇, R₈, R₉, R₁₀, R₁₁, R₁₂, R₁₃, and R₁₄ is deuterium; and

when R₃, R₄, and R₅ are deuterium then at least one of R₁, R₂, R₆, R₇, R₈, R₉, R₁₀, R₁₁, R₁₂, R₁₃, and R₁₄ is deuterium.

In a further embodiment, said compound is substantially a single enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, substantially an individual diastereomer, or a mixture of about 90% or more by weight of an individual diastereomer and about 10% or less by weight of any other diastereomer.

In another embodiment, at least one R₁, R₂, R₃, R₄, R₅, R₆, R₇, R₈, R₉, R₁₀, R₁₁, R₁₂, R₁₃, and R₁₄ independently has deuterium enrichment of no less than about 1%, no less than about 5%, no less than about 10%, no less than about 20%, no less than about 50%, no less than about 70%, no less than about 80%, no less than about 90%, or no less than about 98%.

In other embodiments, R₁ is hydrogen. In yet other embodiments, R₂ is hydrogen. In still other embodiments, R₃ is hydrogen. In yet other embodiments, R₄ is hydrogen. In still other embodiments, R₅ is hydrogen. In yet other embodiments, R₆ is hydrogen. In still other embodiments, R₇ is hydrogen. In still other embodiments, R₈ is hydrogen. In some embodiments, R₉ is hydrogen. In other embodiments, R₁₀ is hydrogen. In yet other embodiments, R₁₁ is hydrogen. In still other embodiments, R₁₂ is hydrogen. In yet other embodiments, R₁₃ is hydrogen. In other embodiments, R₁₄ is hydrogen.

In other embodiments, R₁ is deuterium. In yet other embodiments, R₂ is deuterium. In still other embodiments, R₃ is deuterium. In yet other embodiments, R₄ is deuterium. In still other embodiments, R₅ is deuterium. In yet other embodiments, R₆ is deuterium. In still other embodiments, R₇ is deuterium. In still other embodiments, R₈ is deuterium. In some embodiments, R₉ is deuterium. In other embodiments, R₁₀ is deuterium. In yet other embodiments, R₁₁ is deuterium. In still other embodiments, R₁₂ is deuterium. In yet other embodiments, R₁₃ is deuterium. In other embodiments, R₁₄ is deuterium.

In yet another embodiment of the invention, there are disclosed compounds according to Formula I having one of the following structures:

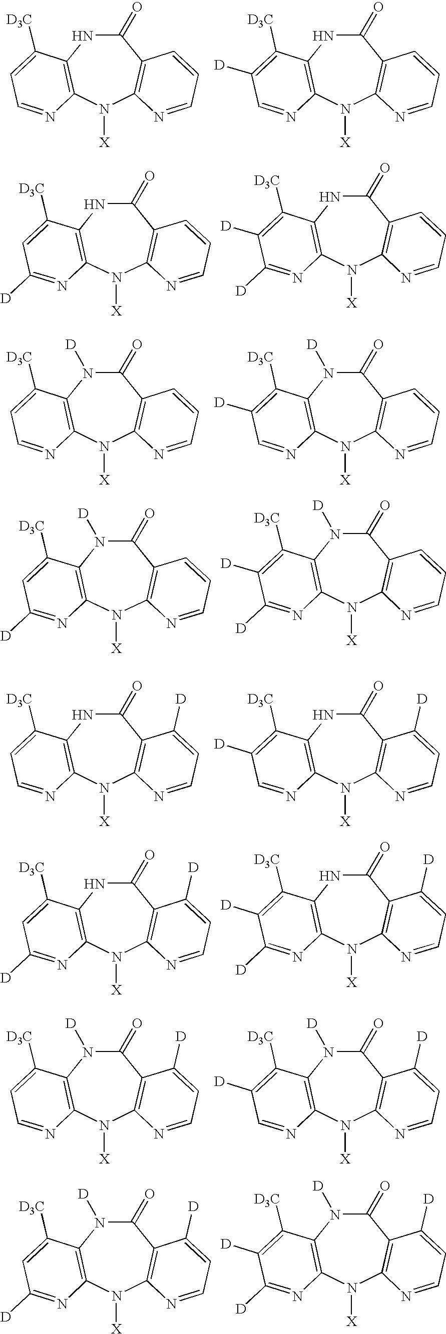

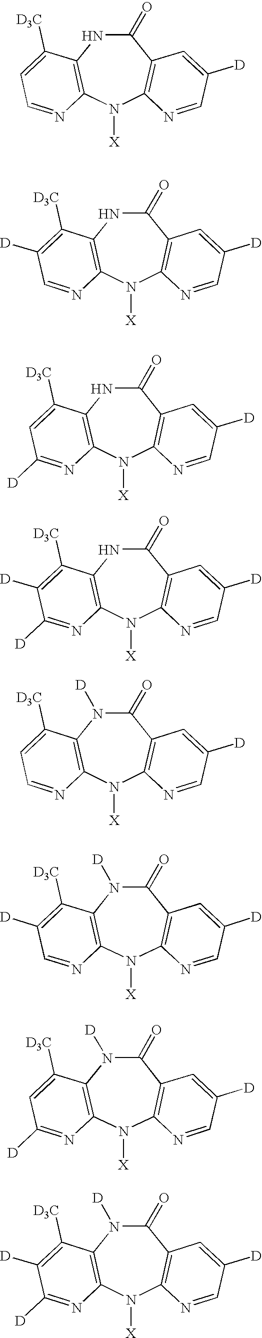

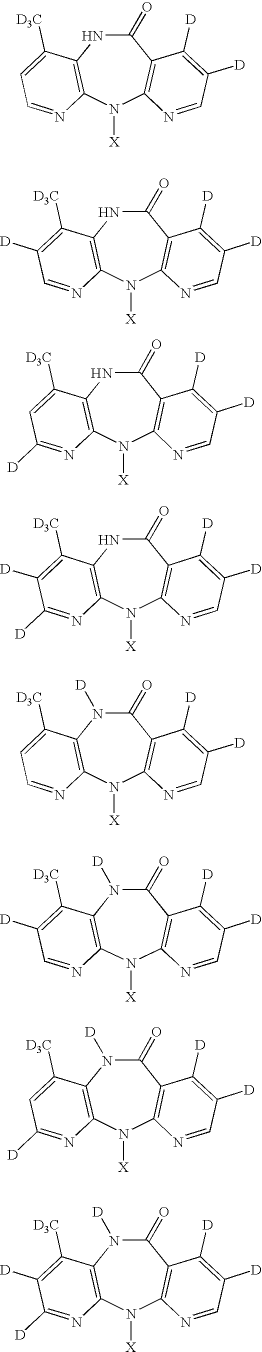

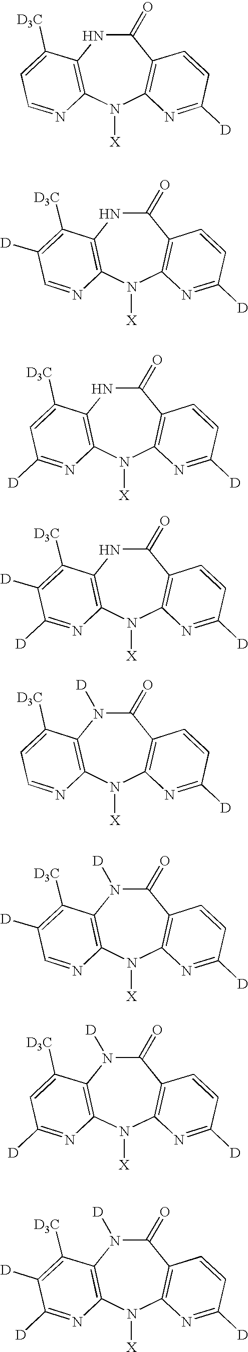

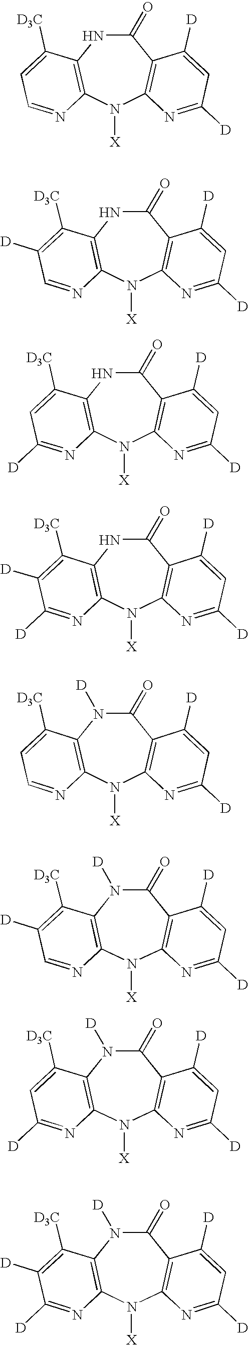

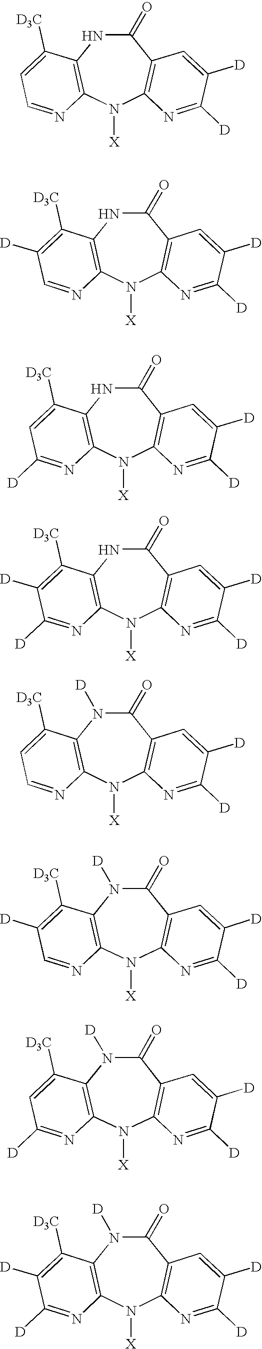

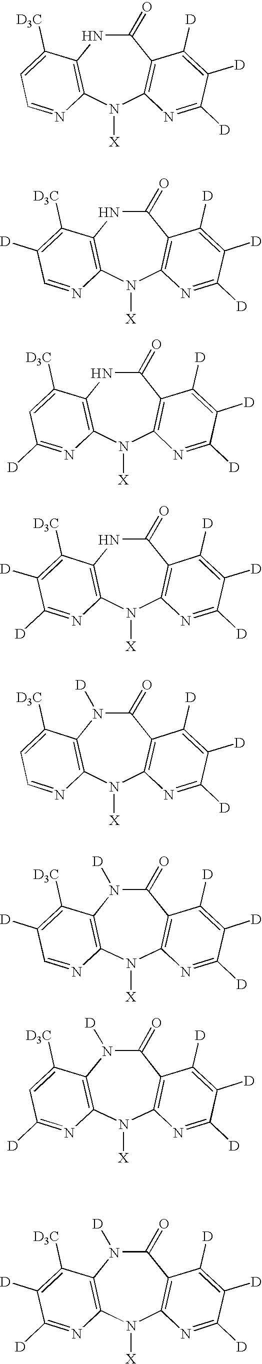

or a pharmaceutically acceptable salt, solvate, or prodrug thereof, wherein X is selected from the group consisting of cyclopropyl, d₁-cyclopropyl, d₂-cyclopropyl, d₃-cyclopropyl, d₄-cyclopropyl, and d₅-cyclopropyl; and with the proviso that the compound cannot be

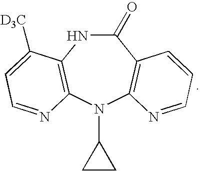

In another embodiment, at least one of the indicated D's independently has deuterium enrichment of no less than about 1%, no less than about 5%, no less than about 10%, no less than about 20%, no less than about 50%, no less than about 70%, no less than about 80%, no less than about 90%, or no less than about 98%.

In a further embodiment, said compound is substantially a single enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, substantially an individual diastereomer, or a mixture of about 90% or more by weight of an individual diastereomer and about 10% or less by weight of any other diastereomer.

In yet another embodiment of the invention, there are disclosed compounds according to Formula I having one of the following structures:

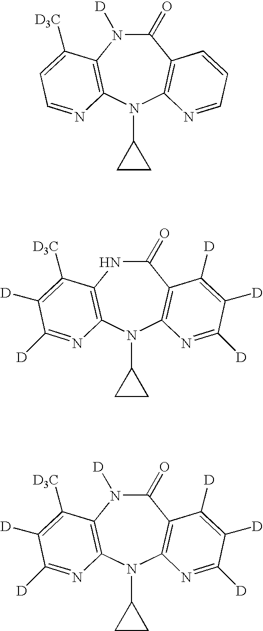

or a pharmaceutically acceptable salt, solvate, or prodrug thereof.

In another embodiment, at least one of the indicated D's independently has deuterium enrichment of no less than about 1%, no less than about 5%, no less than about 10%, no less than about 20%, no less than about 50%, no less than about 70%, no less than about 80%, no less than about 90%, or no less than about 98%.

In a further embodiment, said compound is substantially a single enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, substantially an individual diastereomer, or a mixture of about 90% or more by weight of an individual diastereomer and about 10% or less by weight of any other diastereomer.

In yet another embodiment of the invention, there is a disclosed compound according to Formula I having the following structure:

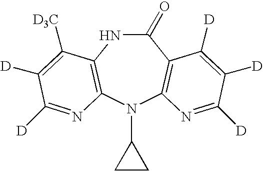

or a pharmaceutically acceptable salt, solvate, or prodrug thereof.

In another embodiment, at least one of the indicated D's independently has deuterium enrichment of no less than about 1%, no less than about 5%, no less than about 10%, no less than about 20%, no less than about 50%, no less than about 70%, no less than about 80%, no less than about 90%, or no less than about 98%.

In a further embodiment, said compound is substantially a single enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, substantially an individual diastereomer, or a mixture of about 90% or more by weight of an individual diastereomer and about 10% or less by weight of any other diastereomer.

In certain embodiments, the compound as disclosed herein contains about 60% or more by weight of the (−)-enantiomer of the compound and about 40% or less by weight of (+)-enantiomer of the compound. In certain embodiments, the compound as disclosed herein contains about 70% or more by weight of the (−)-enantiomer of the compound and about 30% or less by weight of (+)-enantiomer of the compound. In certain embodiments, the compound as disclosed herein contains about 80% or more by weight of the (−)-enantiomer of the compound and about 20% or less by weight of (+)-enantiomer of the compound. In certain embodiments, the compound as disclosed herein contains about 90% or more by weight of the (−)-enantiomer of the compound and about 10% or less by weight of the (+)-enantiomer of the compound. In certain embodiments, the compound as disclosed herein contains about 95% or more by weight of the (−)-enantiomer of the compound and about 5% or less by weight of (+)-enantiomer of the compound. In certain embodiments, the compound as disclosed herein contains about 99% or more by weight of the (−)-enantiomer of the compound and about 1% or less by weight of (+)-enantiomer of the compound.

In certain embodiments, the compound as disclosed herein contains about 60% or more by weight of the (+)-enantiomer of the compound and about 40% or less by weight of (−)-enantiomer of the compound. In certain embodiments, the compound as disclosed herein contains about 70% or more by weight of the (+)-enantiomer of the compound and about 30% or less by weight of (−)-enantiomer of the compound. In certain embodiments, the compound as disclosed herein contains about 80% or more by weight of the (+)-enantiomer of the compound and about 20% or less by weight of (−)-enantiomer of the compound. In certain embodiments, the compound as disclosed herein contains about 90% or more by weight of the (+)-enantiomer of the compound and about 10% or less by weight of the (−)-enantiomer of the compound. In certain embodiments, the compound as disclosed herein contains about 95% or more by weight of the (+)-enantiomer of the compound and about 5% or less by weight of (−)-enantiomer of the compound. In certain embodiments, the compound as disclosed herein contains about 99% or more by weight of the (+)-enantiomer of the compound and about 1% or less by weight of (−)-enantiomer of the compound.

The deuterated compound as disclosed herein may also contain less prevalent isotopes of other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen.

In certain embodiments, without being bound by any theory, the compound disclosed herein may expose a patient to a maximum of about 0.000005% D₂O or about 0.00001% DHO, assuming that all of the C—D bonds in the compound as disclosed herein are metabolized and released as D₂O or DHO. This quantity is a small fraction of the naturally occurring background levels of D₂O or DHO in circulation. In certain embodiments, the levels of D₂O shown to cause toxicity in animals is far greater than the maximally achieved exposure dose of the deuterium enriched compounds disclosed herein. Thus, in certain embodiments, the deuterium-enriched compound disclosed herein should not cause any additional toxicity because of the use of deuterium.

In one embodiment, the deuterated compounds disclosed herein maintain the beneficial aspects of the corresponding non-isotopically enriched molecules while substantially increasing the maximum tolerated dose, decreasing toxicity, increasing the half-life (T_{1/2}), lowering the maximum plasma concentration (Cₘₐₓ) of the minimum efficacious dose (MED), lowering the efficacious dose and thus decreasing the non-mechanism-related toxicity, and/or lowering the probability of drug-drug interactions.

Isotopic hydrogen can be introduced into a compound as disclosed herein by synthetic techniques that employ deuterated reagents, whereby incorporation rates are pre-determined; and/or by exchange techniques, wherein incorporation rates are determined by equilibrium conditions, and may be highly variable depending on the reaction conditions. Synthetic techniques, where tritium or deuterium is directly and specifically inserted by tritiated or deuterated reagents of known isotopic content, may yield high tritium or deuterium abundance, but can be limited by the chemistry required. Exchange techniques, on the other hand, may yield lower tritium or deuterium incorporation, often with the isotope being distributed over many sites on the molecule.

The compounds as disclosed herein can be prepared by methods known to one of skill in the art and routine modifications thereof, and/or following procedures similar to those described in the Example section herein and routine modifications thereof, and/or procedures found in Sommers et al, Journal of the American Chemical Society 1954, 76, 1187-1188; Brickner, J. Med. Chem. 1996, 39, 673-679; Lu, Organic Process Research & Development 2006, 10, 272-277; Tangallapally et al, Journal of Medicinal Chemistry 2005, 48(26), 8261-8269; Perrault, Organic Process Research & Development 2003, 7(4), 533-546 and references cited therein and routine modifications thereof. Compounds as disclosed herein can also be prepared as shown in any of the following schemes and routine modifications thereof.

For example, certain compounds as disclosed herein can be prepared as shown in Scheme 1.

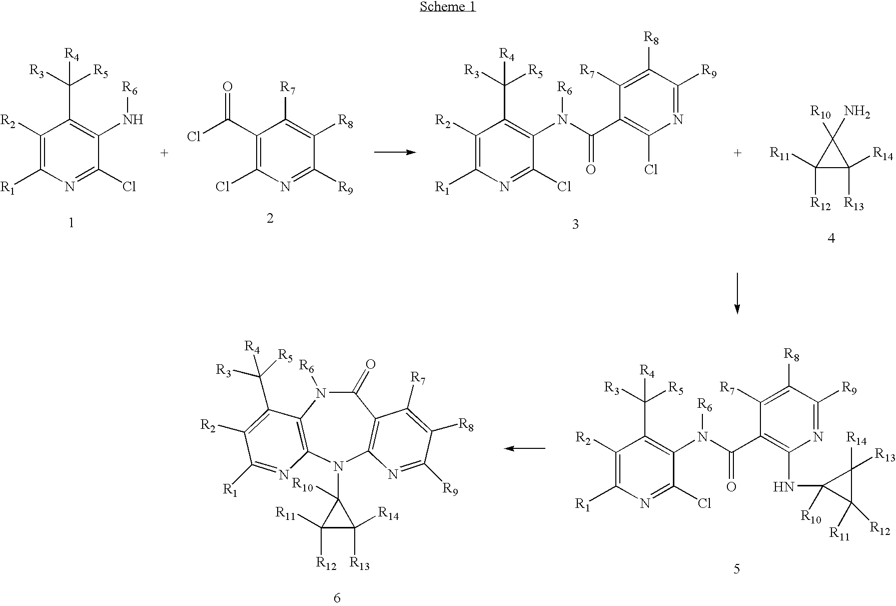

Amino-pyridine 1 is reacted with acyl chloride 2 in appropriate solvent, such as acetonitrile, in the presence of an appropriate base, such as pyridine, at an elevated temperature, to generate chloro-nicotinamide 3. Compound 3 is then reacted with amine 4, in the presence of a basic anhydride, such as calcium oxide, in an appropriate solvent, such as xylene, at an elevated temperature, to afford cyclopropylamino 5. Compound 5 is then treated with a coupling agent, such as activated cuprous iodide, in an inert atmosphere, such as nitrogen, in an appropriate solvent, such as 1-methoxy-2-(2-methoxyethoxy)ethane, at an elevated temperature, to generate compound 6 of Formula I.

Deuterium can be incorporated to different positions synthetically, according to the synthetic procedures as shown in Scheme 1, by using appropriate deuterated intermediates. For example, to introduce deuterium at one or more positions of R₁₀, R₁₁, R₁₂, R₁₃ and R₁₄, cyclopropylamine with the corresponding deuterium substitutions can be used. These deuterated intermediates are either commercially available, or can be prepared by methods known to one of skill in the art or following procedures similar to those described in the Example section herein and routine modifications thereof.

Deuterium can also be incorporated to various positions having an exchangeable proton, such as the amide N—H, methyl C—Hs and pyridine C—Hs via proton-deuterium equilibrium exchange. For example, to introduce deuterium at R₁, R₂, R₃, R₄, R₆, R₅, R₇, R₈, and R₉, the proton may be replaced with deuterium selectively or non-selectively through a proton-deuterium exchange method known in the art.

It is to be understood that the compounds disclosed herein may contain one or more chiral centers, chiral axes, and/or chiral planes, as described in “Stereochemistry of Carbon Compounds” Eliel and Wilen, John Wiley & Sons, New York, 1994, pp. 1119-1190. Such chiral centers, chiral axes, and chiral planes may be of either the (R) or (S) configuration, or may be a mixture thereof.

Another method for characterizing a composition containing a compound having at least one chiral center is by the effect of the composition on a beam of polarized light. When a beam of plane polarized light is passed through a solution of a chiral compound, the plane of polarization of the light that emerges is rotated relative to the original plane. This phenomenon is known as optical activity, and compounds that rotate the plane of polarized light are said to be optically active. One enantiomer of a compound will rotate the beam of polarized light in one direction, and the other enantiomer will rotate the beam of light in the opposite direction. The enantiomer that rotates the polarized light in the clockwise direction is the (+) enantiomer and the enantiomer that rotates the polarized light in the counterclockwise direction is the (−) enantiomer. Included within the scope of the compositions described herein are compositions containing between 0 and 100% of the (+) and/or (−) enantiomer of compounds as disclosed herein.

Where a compound as disclosed herein contains an alkenyl or alkenylene group, the compound may exist as one or mixture of geometric cis/trans (or Z/E) isomers. Where structural isomers are interconvertible via a low energy barrier, the compound as disclosed herein may exist as a single tautomer or a mixture of tautomers. This can take the form of proton tautomerism in the compound as disclosed herein that contains for example, an imino, keto, or oxime group; or so-called valence tautomerism in the compound that contain an aromatic moiety. It follows that a single compound may exhibit more than one type of isomerism.

The compounds disclosed herein may be enantiomerically pure, such as a single enantiomer or a single diastereomer, or be stereoisomeric mixtures, such as a mixture of enantiomers, a racemic mixture, or a diastereomeric mixture. As such, one of skill in the art will recognize that administration of a compound in its (R) form is equivalent, for compounds that undergo epimerization in vivo, to administration of the compound in its (S) form. Conventional techniques for the preparation/isolation of individual enantiomers include chiral synthesis from a suitable optically pure precursor or resolution of the racemate using, for example, chiral chromatography, recrystallization, resolution, diastereomeric salt formation, or derivatization into diastereomeric adducts followed by separation.

When the compound as disclosed herein contains an acidic or basic moiety, the compound may also be embodied as a pharmaceutically acceptable salt (See, Berge et al., J. Pharm. Sci. 1977, 66, 1-19; and “Handbook of Pharmaceutical Salts, Properties, and Use,” Stah and Wermuth, Ed.; Wiley-VCH and VHCA, Zurich, 2002).

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

The compound as disclosed herein may also be designed as a prodrug, which is a functional derivative of the compound as disclosed herein and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis. See Harper, Progress in Drug Research 1962, 4, 221-294; Morozowich et al. in “Design of Biopharmaceutical Properties through Prodrugs and Analogs,” Roche Ed., APHA Acad. Pharm. Sci. 1977; “Bioreversible Carriers in Drug in Drug Design, Theory and Application,” Roche Ed., APHA Acad. Pharm. Sci. 1987; “Design of Prodrugs,” Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asghamejad in “Transport Processes in Pharmaceutical Systems,” Amidon et al., Ed., Marcell Dekker, 185-218, 2000; Balant et al., Eur. J. Drug Metab. Pharmacokinet. 1990, 15, 143-53; Balimane and Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev. 1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72, 324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; Waller et al., Br. J. Clin. Pharmac. 1989, 28, 497-507.

### Pharmaceutical Composition

Disclosed herein are pharmaceutical compositions comprising a compound as disclosed herein as an active ingredient, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof, or a pharmaceutically acceptable salt, solvate, or prodrug thereof, in a pharmaceutically acceptable vehicle, carrier, diluent, or excipient, or a mixture thereof; in combination with one or more pharmaceutically acceptable excipients or carriers.

Disclosed herein are pharmaceutical compositions in modified release dosage forms, which comprise a compound as disclosed herein, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof, or a pharmaceutically acceptable salt, solvate, or prodrug thereof; and one or more release controlling excipients or carriers as described herein. Suitable modified release dosage vehicles include, but are not limited to, hydrophilic or hydrophobic matrix devices, water-soluble separating layer coatings, enteric coatings, osmotic devices, multiparticulate devices, and combinations thereof. The pharmaceutical compositions may also comprise non-release controlling excipients or carriers.

Further disclosed herein are pharmaceutical compositions in enteric coated dosage forms, which comprise a compound as disclosed herein, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof, or a pharmaceutically acceptable salt, solvate, or prodrug thereof, and one or more release controlling excipients or carriers for use in an enteric coated dosage form. The pharmaceutical compositions may also comprise non-release controlling excipients or carriers.

Further disclosed herein are pharmaceutical compositions in effervescent dosage forms, which comprise a compound as disclosed herein, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof, or a pharmaceutically acceptable salt, solvate, or prodrug thereof, and one or more release controlling excipients or carriers for use in an enteric coated dosage form. The pharmaceutical compositions may also comprise non-release controlling excipients or carriers.

Additionally disclosed are pharmaceutical compositions in a dosage form that has an instant releasing component and at least one delayed releasing component, and is capable of giving a discontinuous release of the compound in the form of at least two consecutive pulses separated in time from 0.1 up to 24 hours. The pharmaceutical compositions comprise a compound as disclosed herein, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof, or a pharmaceutically acceptable salt, solvate, or prodrug thereof, and one or more release controlling and non-release controlling excipients or carriers, such as those excipients or carriers suitable for a disruptable semi-permeable membrane and as swellable substances.

Disclosed herein also are pharmaceutical compositions in a dosage form for oral administration to a subject, which comprise a compound as disclosed herein, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof, or a pharmaceutically acceptable salt, solvate, or prodrug thereof, and one or more pharmaceutically acceptable excipients or carriers, enclosed in an intermediate reactive layer comprising a gastric juice-resistant polymeric layered material partially neutralized with alkali and having cation exchange capacity and a gastric juice-resistant outer layer.

Disclosed herein also are pharmaceutical compositions in a dosage form for oral administration to a subject, which comprise a compound as disclosed herein, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof, or a pharmaceutically acceptable salt, solvate, or prodrug thereof, and one or more pharmaceutically acceptable excipients or carriers, formulated as flavored granules that can be reconstituted in water, juice, or the like.

Disclosed herein are pharmaceutical compositions that comprise about 0.1 to about 1000 mg, about 1 to about 500 mg, about 2 to about 100 mg, about 1 mg, about 2 mg, about 3 mg, about 5 mg, about 10 mg, about 20 mg, about 30 mg, about 40 mg, about 50 mg, about 100 mg, about 200 mg, about 400 mg, about 500 mg, about 600 mg, about 1000 mg of one or more compounds disclosed herein in the form tablets for oral administration. The pharmaceutical compositions further comprise inactive ingredients such as cellulose, lactose monohydrate, povidone, sodium starch glycolate, colloidal silicon dioxide and magnesium stearate.

Disclosed herein are pharmaceutical compositions that comprise about 0.1 to about 1000 mg, about 1 to about 500 mg, about 2 to about 100 mg, about 1 mg, about 2 mg, about 3 mg, about 5 mg, about 10 mg, about 20 mg, about 30 mg, about 40 mg, about 50 mg, about 100 mg, about 400 mg, about 500 mg, about 600 mg, about 1000 mg of one or more compounds disclosed herein in the form a suspension for oral administration. The pharmaceutical compositions further comprise inactive ingredients such as carbomer 934P, methylparaben, propylparaben, sorbitol, sucrose, polysorbate 80, sodium hydroxide and purified water.

The pharmaceutical compositions disclosed herein may be disclosed in unit-dosage forms or multiple-dosage forms. Unit-dosage forms, as used herein, refer to physically discrete units suitable for administration to human and animal subjects and packaged individually as is known in the art. Each unit-dose contains a predetermined quantity of the active ingredient(s) sufficient to produce the desired therapeutic effect, in association with the required pharmaceutical carriers or excipients. Examples of unit-dosage forms include ampoules, syringes, and individually packaged tablets and capsules. Unit-dosage forms may be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules, or bottles of pints or gallons.

The compounds disclosed herein may be administered alone, or in combination with one or more other compounds disclosed herein, one or more other active ingredients. The pharmaceutical compositions that comprise a compound disclosed herein may be formulated in various dosage forms for oral, parenteral, and topical administration. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Deliver Technology, Rathbone et al., Eds., Drugs and the Pharmaceutical Science, Marcel Dekker, Inc.: New York, N.Y., 2002; Vol. 126).

The pharmaceutical compositions disclosed herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disorder.

In the case wherein the patient's status does improve, upon the doctor's discretion the administration of the compounds may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disorder is retained. Patients can, however, require intermittent treatment on a long-term basis upon any recurrence of symptoms.

### A. Oral Administration

The pharmaceutical compositions disclosed herein may be disclosed in solid, semisolid, or liquid dosage forms for oral administration. As used herein, oral administration also include buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 50 to about 99% by weight in the pharmaceutical compositions disclosed herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions disclosed herein varies upon the type of formulation, and is readily discernible to those of ordinary skill in the art. The pharmaceutical compositions disclosed herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions disclosed herein may contain about 0.1 to about 5% by weight of a lubricant.

Suitable glidants include colloidal silicon dioxide, CAB-O-SIL® (Cabot Co. of Boston, Mass.), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many carriers and excipients may serve several functions, even within the same formulation.

The pharmaceutical compositions disclosed herein may be disclosed as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions disclosed herein may be disclosed as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms disclosed herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The pharmaceutical compositions disclosed herein may be disclosed in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) disclosed herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions disclosed herein for oral administration may be also disclosed in the forms of liposomes, micelles, microspheres, or nanosystems. Micellar dosage forms can be prepared as described in U.S. Pat. No. 6,350,458.

The pharmaceutical compositions disclosed herein may be disclosed as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions disclosed herein may be formulated as immediate or modified release dosage forms, including delayed-, sustained, pulsed-, controlled, targeted-, and programmed-release forms.

The pharmaceutical compositions disclosed herein may be co-formulated with other active ingredients which do not impair the desired therapeutic action, or with substances that supplement the desired action, such as drotrecogin-α, and hydrocortisone.

### B. Parenteral Administration

The pharmaceutical compositions disclosed herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

The pharmaceutical compositions disclosed herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, dimethylacetamide, and dimethylsulfoxide.

Suitable antimicrobial agents or preservatives include, but are not limited to, phenols, cresols, mercurials, benzyl alcohol, chlorobutanol, methyl and propyl p-hydroxybenzates, thimerosal, benzalkonium chloride, benzethonium chloride, methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

The pharmaceutical compositions disclosed herein may be formulated for single or multiple dosage administration. The single dosage formulations are packaged in an ampule, a vial, or a syringe. The multiple dosage parenteral formulations must contain an antimicrobial agent at bacteriostatic or fungistatic concentrations. All parenteral formulations must be sterile, as known and practiced in the art.

In one embodiment, the pharmaceutical compositions are disclosed as ready-to-use sterile solutions. In another embodiment, the pharmaceutical compositions are disclosed as sterile dry soluble products, including lyophilized powders and hypodermic tablets, to be reconstituted with a vehicle prior to use. In yet another embodiment, the pharmaceutical compositions are disclosed as ready-to-use sterile suspensions. In yet another embodiment, the pharmaceutical compositions are disclosed as sterile dry insoluble products to be reconstituted with a vehicle prior to use. In still another embodiment, the pharmaceutical compositions are disclosed as ready-to-use sterile emulsions.

The pharmaceutical compositions disclosed herein may be formulated as immediate or modified release dosage forms, including delayed-, sustained, pulsed-, controlled, targeted-, and programmed-release forms.

The pharmaceutical compositions may be formulated as a suspension, solid, semi-solid, or thixotropic liquid, for administration as an implanted depot. In one embodiment, the pharmaceutical compositions disclosed herein are dispersed in a solid inner matrix, which is surrounded by an outer polymeric membrane that is insoluble in body fluids but allows the active ingredient in the pharmaceutical compositions diffuse through.

Suitable inner matrixes include polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers, such as hydrogels of esters of acrylic and methacrylic acid, collagen, cross-linked polyvinylalcohol, and cross-linked partially hydrolyzed polyvinyl acetate.

Suitable outer polymeric membranes include polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### C. Topical Administration

The pharmaceutical compositions disclosed herein may be administered topically to the skin, orifices, or mucosa. The topical administration, as used herein, include (intra)dermal, conjuctival, intracorneal, intraocular, ophthalmic, auricular, transdermal, nasal, vaginal, uretheral, respiratory, and rectal administration.

The pharmaceutical compositions disclosed herein may be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation of the pharmaceutical compositions disclosed herein may also comprise liposomes, micelles, microspheres, nanosystems, and mixtures thereof.

Pharmaceutically acceptable carriers and excipients suitable for use in the topical formulations disclosed herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryopretectants, lyoprotectants, thickening agents, and inert gases.

The pharmaceutical compositions may also be administered topically by electroporation, iontophoresis, phonophoresis, sonophoresis and microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions disclosed herein may be disclosed in the forms of ointments, creams, and gels. Suitable ointment vehicles include oleaginous or hydrocarbon vehicles, including such as lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include crosslinked acrylic acid polymers, such as carbomers, carboxypolyalkylenes, Carbopol®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions disclosed herein may be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with the pharmaceutical compositions disclosed herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydrogels, such as polyvinyl alcohol, hydroxyethyl methacrylate, polyacrylic acid; glycerinated gelatin. Combinations of the various vehicles may be used. Rectal and vaginal suppositories may be prepared by the compressed method or molding. The typical weight of a rectal and vaginal suppository is about 2 to about 3 g.

The pharmaceutical compositions disclosed herein may be administered ophthalmically in the forms of solutions, suspensions, ointments, emulsions, gel-forming solutions, powders for solutions, gels, ocular inserts, and implants.

The pharmaceutical compositions disclosed herein may be administered intranasally or by inhalation to the respiratory tract. The pharmaceutical compositions may be disclosed in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions may also be disclosed as a dry powder for insufflation, alone or in combination with an inert carrier such as lactose or phospholipids; and nasal drops. For intranasal use, the powder may comprise a bioadhesive agent, including chitosan or cyclodextrin.

Solutions or suspensions for use in a pressurized container, pump, spray, atomizer, or nebulizer may be formulated to contain ethanol, aqueous ethanol, or a suitable alternative agent for dispersing, solubilizing, or extending release of the active ingredient disclosed herein, a propellant as solvent; and/or a surfactant, such as sorbitan trioleate, oleic acid, or an oligolactic acid.

The pharmaceutical compositions disclosed herein may be micronized to a size suitable for delivery by inhalation, such as about 50 micrometers or less, or about 10 micrometers or less. Particles of such sizes may be prepared using a comminuting method known to those skilled in the art, such as spiral jet milling, fluid bed jet milling, super critical fluid processing to form nanoparticles, high pressure homogenization, or spray drying.

Capsules, blisters and cartridges for use in an inhaler or insufflator may be formulated to contain a powder mix of the pharmaceutical compositions disclosed herein; a suitable powder base, such as lactose or starch; and a performance modifier, such as l-leucine, mannitol, or magnesium stearate. The lactose may be anhydrous or in the form of the monohydrate. Other suitable excipients or carriers include dextran, glucose, maltose, sorbitol, xylitol, fructose, sucrose, and trehalose. The pharmaceutical compositions disclosed herein for inhaled/intranasal administration may further comprise a suitable flavor, such as menthol and levomenthol, or sweeteners, such as saccharin or saccharin sodium.

The pharmaceutical compositions disclosed herein for topical administration may be formulated to be immediate release or modified release, including delayed-, sustained-, pulsed-, controlled-, targeted, and programmed release.

### D. Modified Release

The pharmaceutical compositions disclosed herein may be formulated as a modified release dosage form. As used herein, the term “modified release” refers to a dosage form in which the rate or place of release of the active ingredient(s) is different from that of an immediate dosage form when administered by the same route. Modified release dosage forms include delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

Examples of modified release include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500.

### 1. Matrix Controlled Release Devices

The pharmaceutical compositions disclosed herein in a modified release dosage form may be fabricated using a matrix controlled release device known to those skilled in the art (see, Takada et al in “Encyclopedia of Controlled Drug Delivery,” Vol. 2, Mathiowitz ed., Wiley, 1999).

In one embodiment, the pharmaceutical compositions disclosed herein in a modified release dosage form is formulated using an erodible matrix device, which is water-swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

In further embodiments, the pharmaceutical compositions are formulated with a non-erodible matrix device. The active ingredient(s) is dissolved or dispersed in an inert matrix and is released primarily by diffusion through the inert matrix once administered. Materials suitable for use as a non-erodible matrix device included, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinylacetate copolymers, ethylene/propylene copolymers, ethylene/ethyl acrylate copolymers, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethyleneterephthalate, natural rubber, silicone rubbers, polydimethylsiloxanes, silicone carbonate copolymers, and; hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

The pharmaceutical compositions disclosed herein in a modified release dosage form may be prepared by methods known to those skilled in the art, including direct compression, dry or wet granulation followed by compression, melt-granulation followed by compression.

### 2. Osmotic Controlled Release Devices

The pharmaceutical compositions disclosed herein in a modified release dosage form may be fabricated using an osmotic controlled release device, including one-chamber system, two-chamber system, asymmetric membrane technology (AMT), and extruding core system (ECS). In general, such devices have at least two components: (a) the core which contains the active ingredient(s); and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents water-swellable hydrophilic polymers, which are also referred to as “osmopolymers” and “hydrogels,” including, but not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents are osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates may be employed to influence how rapidly the active ingredient(s) is initially delivered from the dosage form. For example, amorphous sugars, such as Mannogeme EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core may also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxlated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane may also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane may be formed post-coating by mechanical or laser drilling. Delivery port(s) may also be formed in situ by erosion of a plug of water-soluble material or by rupture of a thinner portion of the membrane over an indentation in the core. In addition, delivery ports may be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The pharmaceutical compositions in an osmotic controlled-release dosage form may further comprise additional conventional excipients or carriers as described herein to promote performance or processing of the formulation.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In certain embodiments, the pharmaceutical compositions disclosed herein are formulated as AMT controlled-release dosage form, which comprises an asymmetric osmotic membrane that coats a core comprising the active ingredient(s) and other pharmaceutically acceptable excipients or carriers. See, U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coating method.

In certain embodiments, the pharmaceutical compositions disclosed herein are formulated as ESC controlled-release dosage form, which comprises an osmotic membrane that coats a core comprising the active ingredient(s), a hydroxylethyl cellulose, and other pharmaceutically acceptable excipients or carriers.

### 3. Multiparticulate Controlled Release Devices

The pharmaceutical compositions disclosed herein in a modified release dosage form may be fabricated a multiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates may be made by the processes know to those skilled in the art, including wet- and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other excipients or carriers as described herein may be blended with the pharmaceutical compositions to aid in processing and forming the multiparticulates. The resulting particles may themselves constitute the multiparticulate device or may be coated by various film-forming materials, such as enteric polymers, water-swellable, and water-soluble polymers. The multiparticulates can be further processed as a capsule or a tablet.

### 4. Targeted Delivery

The pharmaceutical compositions disclosed herein may also be formulated to be targeted to a particular tissue, receptor, or other area of the body of the subject to be treated, including liposome-, resealed erythrocyte-, and antibody-based delivery systems. Examples include, but are not limited to, U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542; and 5,709,874.

Disclosed are methods for treating, preventing, or ameliorating one or more symptoms of a viral-mediated disorder comprising administering to a subject having or being suspected to have such a disorder a therapeutically effective amount of a compound as disclosed herein or a pharmaceutically acceptable salt, solvate, or prodrug thereof.

Viral-mediated disorders include, but are not limited to, infectious disorders and/or any disorder ameliorated by administering an antiinfective agent. In some embodiments the infectious disorder is HIV.

Also disclosed are methods of treating, preventing, or ameliorating one or more symptoms of a viral-mediated disorder, by administering to a subject having or being suspected to have such a disorder, a therapeutically effective amount of a compound as disclosed herein or a pharmaceutically acceptable salt, solvate, or prodrug thereof.

Further disclosed are methods of treating, preventing, or ameliorating one or more symptoms of a disorder responsive to administering a antiinfective agent, comprising administering to a subject having or being suspected to have such a disorder, a therapeutically effective amount of a compound as disclosed herein or a pharmaceutically acceptable salt, solvate, or prodrug thereof.

Furthermore, disclosed herein are methods of modulating the activity of viruses, comprising contacting the virus with at least one compound as disclosed herein or a pharmaceutically acceptable salt, solvate, or prodrug thereof. In one embodiment, the virus is present in a subject's body.

In some embodiments, the disorder involving, but not limited to, infectious disorders and/or any disorder ameliorated by administering an antiinfective agent, is caused by a retrovirus. In another embodiment, the retrovirus is a lentivirus. In further embodiments, the lentivirus is HIV type 1.

Disclosed herein are methods for treating a subject, including a human, having or suspected of having a disorder, involving, but not limited to, infectious disorders and/or any disorder ameliorated by administering a antiinfective agent; comprising administering to the subject a therapeutically effective amount of a compound as disclosed herein or a pharmaceutically acceptable salt, solvate, or prodrug thereof, so as to affect decreased inter-individual variation in plasma levels of said compound or a metabolite thereof during treatment of the above-mentioned disorder as compared to the non-isotopically enriched compound.

In certain embodiments, the inter-individual variation in plasma levels of the compounds as disclosed herein, or metabolites thereof, is decreased by greater than about 5%, greater than about 10%, greater than about 20%, greater than about 30%, greater than about 40%, or by greater than about 50% as compared to the corresponding non-isotopically enriched compound.

Disclosed herein are methods for treating a subject, including a human, having or suspected of having a disorder involving, but not limited to, infectious disorders and/or any disorder ameliorated by administering a antiinfective agent, or for preventing such disorder, in a subject prone to the disorder; comprising administering to the subject a therapeutically effective amount of a compound as disclosed herein or a pharmaceutically acceptable salt, solvate, or prodrug thereof, so as to affect increased average plasma levels of said compound or decreased average plasma levels of at least one metabolite of said compound per dosage unit as compared to the non-isotopically enriched compound.

In certain embodiments, the average plasma levels of the compound as disclosed herein are increased by greater than about 5%, greater than about 10%, greater than about 20%, greater than about 30%, greater than about 40%, or greater than about 50% as compared to the corresponding non-isotopically enriched compounds.

In certain embodiments, the average plasma levels of a metabolite of the compound as disclosed herein are decreased by greater than about 5%, greater than about 10%, greater than about 20%, greater than about 30%, greater than about 40%, or greater than about 50% as compared to the corresponding non-isotopically enriched compounds.

Plasma levels of the compounds as disclosed herein, or metabolites thereof, may be measured using the methods described by Li et al. (Rapid Communications in Mass Spectrometry 2005, 19, 1943-1950).

Disclosed herein are methods for treating a subject, including a human, having or suspected of having a disorder involving, but not limited to, infectious disorders and/or any disorder ameliorated by administering a antiinfective agent, or for preventing such disorder, in a subject prone to the disorder; comprising administering to the subject a therapeutically effective amount of a compound as disclosed herein or a pharmaceutically acceptable salt, solvate, or prodrug thereof, so as to affect a decreased inhibition of, and/or metabolism by at least one cytochrome P₄₅₀ or monoamine oxidase isoform in the subject during the treatment of the disorder as compared to the corresponding non-isotopically enriched compound.

Examples of cytochrome P₄₅₀ isoforms in a mammalian subject include, but are not limited to, CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4×1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51.

Examples of monoamine oxidase isoforms in a mammalian subject include, but are not limited to, MAO_{A}, and MAO_{B}.

In certain embodiments, the decrease in inhibition of the cytochrome P₄₅₀ or monoamine oxidase isoform by a compound as disclosed herein is greater than about 5%, greater than about 10%, greater than about 20%, greater than about 30%, greater than about 40%, or greater than about 50% as compared to the corresponding non-isotopically enriched compounds.

The inhibition of the cytochrome P₄₅₀ isoform is measured by the method of Ko et al. (British Journal of Clinical Pharmacology, 2000, 49, 343-351). The inhibition of the MAO_{A} isoform is measured by the method of Weyler et al. (J. Biol. Chem. 1985, 260, 13199-13207). The inhibition of the MAO_{B} isoform is measured by the method of Uebelhack et al. (Pharmacopsychiatry, 1998, 31, 187-192).

Disclosed herein are methods for treating a subject, including a human, having or suspected of having a disorder involving, but not limited to, infectious disorders and/or any disorder ameliorated by administering a antiinfective agent, or for preventing such disorder, in a subject prone to the disorder; comprising administering to the subject a therapeutically effective amount of a compound as disclosed herein or a pharmaceutically acceptable salt, solvate, or prodrug thereof, so as to affect a decreased metabolism via at least one polymorphically-expressed cytochrome P₄₅₀ isoform in the subject during the treatment of the disorder as compared to the corresponding non-isotopically enriched compound.

Examples of polymorphically-expressed cytochrome P₄₅₀ isoforms in a mammalian subject include, but are not limited to, CYP2C8, CYP2C9, CYP2C19, and CYP2D6.

In certain embodiments, the decrease in metabolism of the compound as disclosed herein by at least one polymorphically-expressed cytochrome P₄₅₀ isoforms cytochrome P₄₅₀ isoform is greater than about 5%, greater than about 10%, greater than about 20%, greater than about 30%, greater than about 40%, or greater than about 50% as compared to the corresponding non-isotopically enriched compound.

The metabolic activities of liver microsomes and the cytochrome P₄₅₀ isoforms are measured by the methods described in Example 4. The metabolic activities of the monoamine oxidase isoforms are measured by the methods described in Examples 5, 6 and 7.

In another aspect of the invention, there are provided methods for treating a subject, particularly a human having, suspected of having, or being prone to a disorder involving, but not limited to, infectious disorders and/or any disorder ameliorated by administering a antiinfective agent, comprising administering to the subject in need thereof a therapeutically effective amount of an antiinfective comprising at least one of the compounds as disclosed herein or a pharmaceutically acceptable salt, solvate, or prodrug thereof, so as to affect prevention or amelioration of infection and/or additional infections as the primary clinical benefit (e.g., maintenance of absence of a disorder, maintenance of absence of additional infections by other viruses) as compared to the non-isotopically enriched compound.

In another embodiment of the invention, there are provided methods for treating a subject, particularly a human having, suspected of having, or being prone to a disorder involving, but not limited to, infectious disorders and/or any disorder ameliorated by administering a antiinfective agent, comprising administering to the subject in need thereof a therapeutically effective amount of an antiinfective agent comprising at least one of the compounds as disclosed herein, or a pharmaceutically acceptable salt, solvate, or prodrug thereof, so as to affect statistically-significantly improved clinical endpoints (e.g., mean reduction of ≧1 log₁₀ copies/mL plasma, etc.) as compared to the non-isotopically enriched compound.

Disclosed herein are methods for treating a subject, including a human, having or suspected of having a disorder involving, but not limited to, infectious disorders and/or any disorder ameliorated by administering a antiinfective agent, or for preventing such disorder, in a subject prone to the disorder; comprising administering to the subject a therapeutically effective amount of a compound as disclosed herein or a pharmaceutically acceptable salt, solvate, or prodrug thereof; so as to affect at least one statistically-significantly improved disorder-control and/or disorder-eradication endpoint, as compared to the corresponding non-isotopically enriched compound.

Examples of improved disorder-control and/or disorder-eradication endpoints include, but are not limited to, a statistically significant increase in CD4 cell count; mean reduction of ≧1 log₁₀ virus copies/mL plasma (viral load); statistically significant decrease in virus RNA levels, disorder progression, mortality, and/or opportunistic infection rates (including bacterial, mycobacterial, viral, protozoan, etc.), as compared to the corresponding non-isotopically enriched compound when given under the same dosing protocol including the same number of doses per day and the same quantity of drug per dose.

Disclosed herein are methods for treating a subject, including a human, having or suspected of having a disorder involving, but not limited to, infectious disorders and/or any disorder ameliorated by administering a antiinfective agent, or for preventing such disorder, in a subject prone to the disorder; comprising administering to the subject a therapeutically effective amount of a compound as disclosed herein or a pharmaceutically acceptable salt, solvate, or prodrug thereof; so as to affect prevention of recurrence, or delay of decline or appearance, of abnormal alimentary or hepatic parameters as the primary clinical benefit, as compared to the corresponding non-isotopically enriched compound.

Disclosed herein are methods for treating a subject, including a human, having or suspected of having a disorder involving, but not limited to, infectious disorders and/or any disorder ameliorated by administering a antiinfective agent, or for preventing such disorder, in a subject prone to the disorder; comprising administering to the subject a therapeutically effective amount of a compound as disclosed herein or a pharmaceutically acceptable salt, solvate, or prodrug thereof, so as to allow the treatment of an infectious disorders and/or any disorder ameliorated by administering a antiinfective agent, while reducing or eliminating deleterious changes in any diagnostic hepatobiliary function endpoints as compared to the corresponding non-isotopically enriched compound.

Examples of diagnostic hepatobiliary function endpoints include, but are not limited to, alanine aminotransferase (“ALT”), serum glutamic-pyruvic transaminase (“SGPT”), aspartate aminotransferase (“AST” or “SGOT”), ALT/AST ratios, serum aldolase, alkaline phosphatase (“ALP”), ammonia levels, bilirubin, gamma-glutamyl transpeptidase (“GGTP,” “γ-GTP,” or “GGT”), leucine aminopeptidase (“LAP”), liver biopsy, liver ultrasonography, liver nuclear scan, 5′-nucleotidase, and blood protein. Hepatobiliary endpoints are compared to the stated normal levels as given in “Diagnostic and Laboratory Test Reference”, 4th edition, Mosby, 1999. These assays are run by accredited laboratories according to standard protocol.

Depending on the disorder to be treated and the subject's condition, the compound as disclosed herein disclosed herein may be administered by oral, parenteral (e.g., intramuscular, intraperitoneal, intravenous, ICV, intracistemal injection or infusion, subcutaneous injection, or implant), inhalation, nasal, vaginal, rectal, sublingual, or topical (e.g., transdermal or local) routes of administration, and may be formulated, alone or together, in suitable dosage unit with pharmaceutically acceptable carriers, adjuvants and vehicles appropriate for each route of administration.

The dose may be in the form of one, two, three, four, five, six, or more sub-doses that are administered at appropriate intervals per day. The dose or sub-doses can be administered in the form of dosage units containing from about 0.1 to about 1000 milligram, from about 0.2 to about 600 milligrams, or from 0.5 about to about 500 milligram active ingredient(s) per dosage unit, and if the condition of the patient requires, the dose can, by way of alternative, be administered as a continuous infusion.

In certain embodiments, an appropriate dosage level is about 0.01 to about 100 mg per kg patient body weight per day (mg/kg per day), about 0.01 to about 50 mg/kg per day, about 0.01 to about 25 mg/kg per day, or about 0.05 to about 10 mg/kg per day, which may be administered in single or multiple doses. A suitable dosage level may be about 0.01 to about 100 mg/kg per day, about 0.05 to about 50 mg/kg per day, or about 0.1 to about 10 mg/kg per day. Within this range the dosage may be about 0.01 to about 0.1, about 0.1 to about 1.0, about 1.0 to about 10, or about 10 to about 50 mg/kg per day.

### Combination Therapy

The compounds disclosed herein may also be combined or used in combination with other agents useful in the treatment, prevention, or amelioration of one or more symptoms of, but not limited to, an infectious disorder, and/or a disorder ameliorated by administering an antiinfective agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced).

Such other agents, adjuvants, or drugs, may be administered, by a route and in an amount commonly used therefor, simultaneously or sequentially with a compound as disclosed herein. When a compound as disclosed herein is used contemporaneously with one or more other drugs, a pharmaceutical composition containing such other drugs in addition to the compound disclosed herein may be utilized, but is not required. Accordingly, the pharmaceutical compositions disclosed herein include those that also contain one or more other active ingredients or therapeutic agents, in addition to the compound disclosed herein.

In certain embodiments, the compounds provided herein can be combined with one or more NRTIs known in the art, including, but not limited to, the group including abacavir, didanosine, emtricitabine, lamivudine, stavudine, zidovudine, tenofovir, apricitabine, stampidine, elvucitabine, racivir and zalcitabine.

In certain embodiments, the compounds provided herein can be combined with one or more NNRTIs known in the art, including, but not limited to, the group including efavirenz, nevirapine, etravirine, rilpivirine, loviride and delavirdine.

In certain embodiments, the compounds provided herein can be combined with one or more protease inhibitors known in the art, including, but not limited to, the group including atazanavir, darunavir, fosamprenavir, lopinavir, nelfinavir, ritonavir, saquinavir, tipranavir, amprenavir and indinavir.

In certain embodiments, the compounds provided herein can be combined with one or more entry/fusion inhibitors known in the art, including, but not limited to, the group including enfuvirtide, maraviroc, PRO 140, and vicriviroc.

In certain embodiments, the compounds provided herein can be combined with one or more integrase inhibitors known in the art, including, but not limited to, the group including raltegravir, and elvitegravir.

In certain embodiments, the compounds provided herein can be combined with one or more maturation inhibitors known in the art, including, but not limited to, the group including bevirimat and vivecon.

In certain embodiments, the compounds provided herein can be combined with one or more antiviral associated agents known in the art, including, but not limited to, the group including foscarnet, chloroquine, quinoline, grapefruit juice, hydroxyurea, leflunomide, mycophenolic acid, resveratrol, ritonavir, epigallocatechin gallate, portmanteau inhibitors, Globoidnan A, griffithsin, diarylpyrimidines, and Calanolide A.

In certain embodiments, the compounds provided herein can be combined with one or more HIV fixed drug combinations known in the art, including, but not limited to, the group including Combivir®, Atripla®, Trizivir®, Truvada®, Kaletra®, and Epzicom®.

In certain embodiments, the compounds provided herein can be combined with one or more antibacterial agents known in the art, including, but not limited to the group including amikacin, p-aminosalisylic acid, amoxicillin, ampicillin, arsphenamine, azithromycin, aztreonam, azlocillin, bacitracin, capreomycin, carbenicillin, cefaclor, cefadroxil, cefamandole, cefazolin, cephalexin, cefdinir, cefditorin, cefepime, cefixime, cefoperazone, cefotaxime, cefoxitin, cefpodoxime, cefprozil, ceftazidime, ceftibuten, ceftizoxime, ceftriaxone, cefuroxime, chloramphenicol, cilastin, ciprofloxacin, clarithromycin, clindamycin, clofazimine, cloxacillin, colistin, cycloserine, dalfopristan, demeclocycline, dicloxacillin, dirithromycin, doxycycline, erythromycin, enafloxacin, enviomycin, ertepenem, ethambutol, ethionamide, flucloxacillin, fosfomycin, furazolidone, gatifloxacin, geldanamycin, gentamicin, herbimicin, imipenem, isoniazide, kanamicin, levofloxacin, linezolid, lomefloxacin, loracarbef, mafenide, moxifloxacin, meropenem, metronidazole, mezlocillin, minocycline, mupirozin, nafcillin, neomycin, netilmicin, nitrofurantoin, norfloxacin, ofloxacin, oxytetracycline, penicillin, piperacillin, platensimycin, polymixin B, prochlorperazine, prontocil, prothionamide, pyrazinamide, quinupristine, rifabutin, rifampin, roxithromycin, spectinomycin, streptomycin, sulfacetamide, sulfamethizole, sulfamethoxazole, teicoplanin, telithromycin, tetracycline, thioacetazone, thioridazine, ticarcillin, tobramycin, trimethoprim, troleandomycin, trovafloxacin, vancomycin and viomycin.

In certain embodiments, the compounds disclosed herein can be combined with one or more antifungal agents known in the art, including, but not limited to the group including amorolfine, amphotericin B, anidulafungin, bifonazole, butenafine, butoconazole, caspofungin, ciclopirox, clotrimazole, econazole, fenticonazole, filipin, fluconazole, isoconazole, itraconazole, ketoconazole, micafungin, miconazole, naftifine, natamycin, nystatin, oxyconazole, ravuconazole, posaconazole, rimocidin, sertaconazole, sulconazole, terbinafine, terconazole, tioconazole, and voriconazole.

In certain embodiments, the compounds disclosed herein can be combined with one or more sepsis treatments known in the art, including, but not limited to drotrecogin-α or a biosimilar of activated protein C.

In certain embodiments, the compounds disclosed herein can be combined with one or more steroidal drugs known in the art, including, but not limited to, aldosterone, beclometasone, betamethasone, deoxycorticosterone acetate, fludrocortisone acetate, hydrocortisone (cortisol), prednisolone, prednisone, methylprenisolone, dexamethasone, and triamcinolone.

In certain embodiments, the compounds disclosed herein can be combined with one or more anticoagulants known in the art, including, but not limited to the group including acenocoumarol, argatroban, bivalirudin, lepirudin, fondaparinux, heparin, phenindione, warfarin, and ximalagatran.

In certain embodiments, the compounds disclosed herein can be combined with one or more thrombolytics known in the art, including, but not limited to the group including anistreplase, reteplase, t-PA (alteplase activase), streptokinase, tenecteplase, and urokinase.

In certain embodiments, the compounds disclosed herein can be combined with one or more non-steroidal anti-inflammatory agents known in the art, including, but not limited to the group including aceclofenac, acemetacin, amoxiprin, aspirin, azapropazone, benorilate, bromfenac, carprofen, celecoxib, choline magnesium salicylate, diclofenac, diflunisal, etodolac, etoracoxib, faislamine, fenbuten, fenoprofen, flurbiprofen, ibuprofen, indometacin, ketoprofen, ketorolac, lomoxicam, loxoprofen, lumiracoxib, meclofenamic acid, mefenamic acid, meloxicam, metamizole, methyl salicylate, magnesium salicylate, nabumetone, naproxen, nimesulide, oxyphenbutazone, parecoxib, phenylbutazone, piroxicam, salicyl salicylate, sulindac, sulfinprazone, suprofen, tenoxicam, tiaprofenic acid, and tolmetin.

In certain embodiments, the compounds disclosed herein can be combined with one or more antiplatelet agents known in the art, including, but not limited to the group including abciximab, cilostazol, clopidogrel, dipyridamole, ticlopidine, and tirofibin.

The compounds disclosed herein can also be administered in combination with other classes of compounds, including, but not limited to, endothelin converting enzyme (ECE) inhibitors, such as phosphoramidon; thromboxane receptor antagonists, such as ifetroban; potassium channel openers; thrombin inhibitors, such as hirudin; growth factor inhibitors, such as modulators of PDGF activity; platelet activating factor (PAF) antagonists; anti-platelet agents, such as GPIIb/IIIa blockers (e.g., abdximab, eptifibatide, and tirofiban), P2Y(AC) antagonists (e.g., clopidogrel, ticlopidine and CS-747), and aspirin; anticoagulants, such as warfarin; low molecular weight heparins, such as enoxaparin; Factor VIIa Inhibitors and Factor Xa Inhibitors; renin inhibitors; neutral endopeptidase (NEP) inhibitors; vasopepsidase inhibitors (dual NEP-ACE inhibitors), such as omapatrilat and gemopatrilat; HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, or atavastatin or visastatin); squalene synthetase inhibitors; fibrates; bile acid sequestrants, such as questran; niacin; anti-atherosclerotic agents, such as ACAT inhibitors; MTP Inhibitors; calcium channel blockers, such as amlodipine besylate; potassium channel activators; alpha-adrenergic agents; beta-adrenergic agents, such as carvedilol and metoprolol; antiarrhythmic agents; diuretics, such as chlorothlazide, hydrochlorothiazide, flumethiazide, hydroflumethiazide, bendroflumethiazide, methylchlorothiazide, trichloromethiazide, polythiazide, benzothlazide, ethacrynic acid, tricrynafen, chlorthalidone, furosenilde, musolimine, bumetanide, triamterene, amiloride, and spironolactone; thrombolytic agents, such as tissue plasminogen activator (tPA), recombinant tPA, streptokinase, urokinase, prourokinase, and anisoylated plasminogen streptokinase activator complex (APSAC); anti-diabetic agents, such as biguanides (e.g. metformin), glucosidase inhibitors (e.g., acarbose), insulins, meglitinides (e.g., repaglinide), sulfonylureas (e.g., glimepiride, glyburide, and glipizide), thiozolidinediones (e.g. troglitazone, rosiglitazone and pioglitazone), and PPAR-gamma agonists; mineralocorticoid receptor antagonists, such as spironolactone and eplerenone; growth hormone secretagogues; aP2 inhibitors; phosphodiesterase inhibitors, such as PDE III inhibitors (e.g., cilostazol) and PDE V inhibitors (e.g., sildenafil, tadalafil, vardenafil); protein tyrosine kinase inhibitors; antiinflammatories; antiproliferatives, such as methotrexate, FK506 (tacrolimus, Prograf), mycophenolate mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes); antimetabolites, such as folate antagonists, purine analogues, and pyrridine analogues; antiinfective s, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone anatagonists, and octreotide acetate; microtubule-disruptor agents, such as ecteinascidins; microtubule-stablizing agents, such as pacitaxel, docetaxel, and epothilones A-F; plant-derived products, such as vinca alkaloids, epipodophyllotoxins, and taxanes; and topoisomerase inhibitors; prenyl-protein transferase inhibitors; and cyclosporins; steroids, such as prednisone and dexamethasone; cytotoxic drugs, such as azathiprine and cyclophosphamide; TNF-alpha inhibitors, such as tenidap; anti-TNF antibodies or soluble TNF receptor, such as etanercept, rapamycin, and leflunimide; and cyclooxygenase-2 (COX-2) inhibitors, such as celecoxib and rofecoxib; and miscellaneous agents such as, hydroxyurea, procarbazine, mitotane, hexamethylmelamine, gold compounds, platinum coordination complexes, such as cisplatin, satraplatin, and carboplatin.

### Kits/Articles of Manufacture

For use in the therapeutic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more compounds described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise a compound with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein. These other therapeutic agents may be used, for example, in the amounts indicated in the Physicians' Desk Reference (PDR) or as otherwise determined by one of ordinary skill in the art.

The invention is further illustrated by the following examples.

### EXAMPLE 1

### d₄-11-Cyclopropyl-5,11-dihydro-4-methyl-6H-dipyrido[3,2,-b:2′,3′-e][1,4]-diazepin-2-one (d₄-Nevirapine)

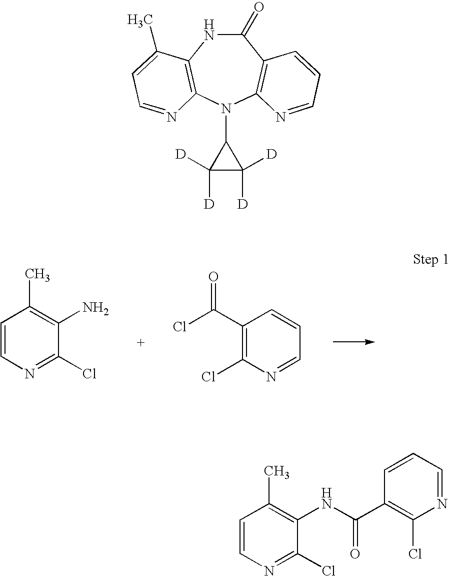

2-Chloro-N-(2-chloro-4-methyl-pyridin-3-yl)-nicotinamide: The procedure is carried out as described in Hargrave J. Med. Chem. 1991, 34, 2231-2241, which is hereby incorporated by reference in its entirety. To a solution of 3-amino-2-chloro-4-methylpyridine (18.2 mmol) in 6:1 cyclohexane-dioxane (6 mL), and pyridine (5.75 mL) is added a solution of 2-chloronicotinoyl chloride (12.8 mmol) in 1,4-dioxane (5 mL). The resulting mixture is stirred at ambient temperature for 48 hours and the precipitate is filtered and washed with water. The solid is taken up in ethanol (17.5 mL) and aqueous NaOH (0.1 N, 3.6 mL). The solution is then heated to reflux for 2 hours, cooled to ambient temperature and stirred overnight. The solvent is removed under vacuum and water (10 mL) is added to residue, with stirring. The mixture is cooled to 10° C. and the crystalline product is filtered, washed with cold water and dried under vacuum to give the desired product, 2-chloro-N-(2-chloro-4-methyl-pyridin-3-yl)-nicotinamide.

### Step 2

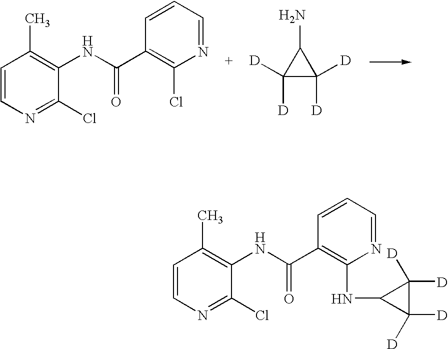

d₄-N-(2-Chloro-4-methylpyridin-3-yl)-2-cyclopropylamino-nicotinamide: The procedure is carried out as described in Hargrave J. Med. Chem. 1991, 34, 2231-2241, which is hereby incorporated by reference in its entirety. A mixture of 2-chloro-N-(2-chloro-4-methyl-pyridin-3-yl)-nicotinamide (5.9 mmol) and d₄-cyclopropylamine (23.5 mmol, C/D/N Isotopes, Pointe-Claire, Quebec, Canada H9R 1H1) in xylene (10 mL) is heated at 110° C., in a sealed tube, for 18 hours. The solution is then cooled to ambient temperature, diluted with dichloromethane (20 mL), washed with water, dried over Na₂SO₄, filtered and concentrated in vacuo. The crude residue is tritrated with dichloromethane and ether to give the desired product, d₄-N-(2-chloro-4-methylpyridin-3-yl)-2-cyclopropylamino-nicotinamide.

### Step 3

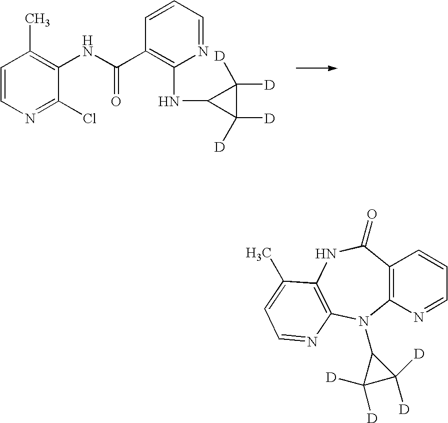

d₄-11-Cyclopropyl-5,11-dihydro-4-methyl-6H-dipyrido[3,2-b:2′,3′-e][1,4]-diazepin-2-one (d₄-Nevirapine): The procedure is carried out as described in Hargrave J. Med. Chem. 1991, 34, 2231-2241, which is hereby incorporated by reference in its entirety. To a solution of d₄-N-(2-chloro-4-methylpyridin-3-yl)-2-cyclopropylamino-nicotinamide (4.8 mmol) in bis(2-methoxyethyl)ether (9 mL) is added NaH (9.6 mmol), under nitrogen. The mixture is heated slowly to 160° C. and then heated under gentle reflux for 1.5 hours. The mixture is cooled to 65° C., poured onto ice-water and stirred overnight. The precipitate is filtered, dissolved in hot pyridine (10 mL). The product is then precipitated by slow addition of water (100 mL). The crude product is isolated by filtration and then redissolved in methanol (10 mL) and refluxed. Filtration followed by recrystallization from pyridine/water gives the desired product, 11-cyclopropyl-5,11-dihydro-4-methyl-6H-dipyrido[3,2,-b:2′,3′-e][1,4]-diazepin-2-one (nevirapine).

### EXAMPLE 2

### d₉-11-Cyclopropyl-5,11-dihydro-4-methyl-6H-dipyrido[3,2,-b:2′,3′-e][1,4]-diazepin-2-one (d₉-Nevirapine)

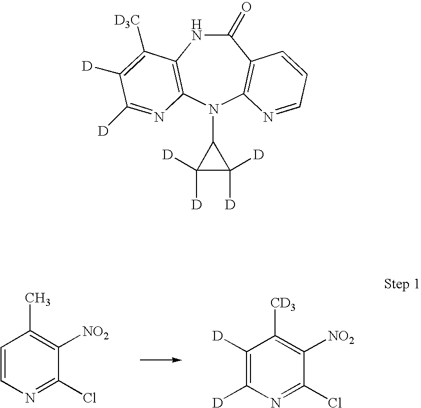

d₅-2-chloro-4-methyl-3-nitropyridine: A mixture of 3-nitro-2-chloro-4-methylpyridine in dioxane and K₂CO₃ in D₂O is heated to 100° C. to give the desired product, d₅-2-chloro-4-methyl-3-nitropyridine.

### Step 2

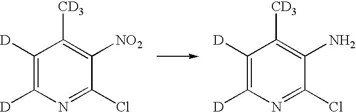

d₅-3-amino-2-chloro-4-methylpyridine: The procedure is carried out as described in Hargrave J. Med. Chem. 1991, 34, 2231-2241, which is hereby incorporated by reference in its entirety. d₅-2-Chloro-4-methyl-3-nitropyridine (12.4 mmol) in ethanol (10 mL) is reduced in the presence of 5% Rh/C, under an atmosphere of hydrogen (50 psi) for 3 hours. The catalyst is filtered through celite and the solvent is removed. The residue is treated with hot ethyl acetate, filtered through celite and concentrated to give the desired product, d₅-3-amino-2-chloro-4-methylpyridine.

### Step 3

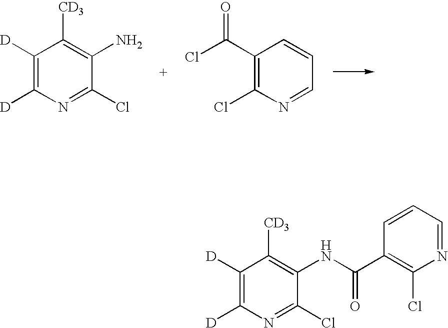

d₅-2-Chloro-N-(2-chloro-4-methyl-pyridin-3-yl)-nicotinamide: The title compound is prepared according to Example 1, step 1, by substituting d₅-3-amino-2-chloro-4-methylpyridine for 3-amino-2-chloro-4-methylpyridine.

### Step 4

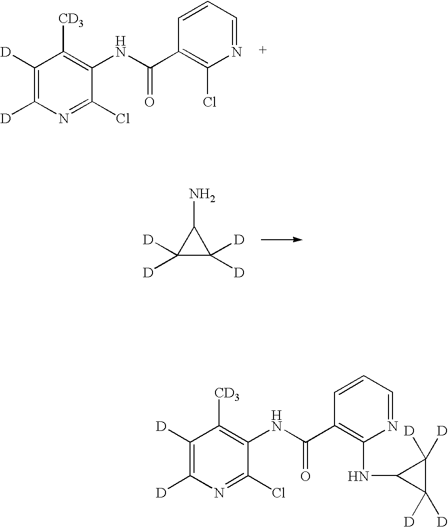

d₉-N-(2-Chloro-4-methylpyridin-3-yl)-2-cyclopropylamino-nicotinamide: The title compound is prepared according to Example 1, step 2, by substituting d₅-2-chloro-N-(2-chloro-4-methyl-pyridin-3-yl)-nicotinamide for 2-chloro-N-(2-chloro-4-methyl-pyridin-3-yl)-nicotinamide.

### Step 5

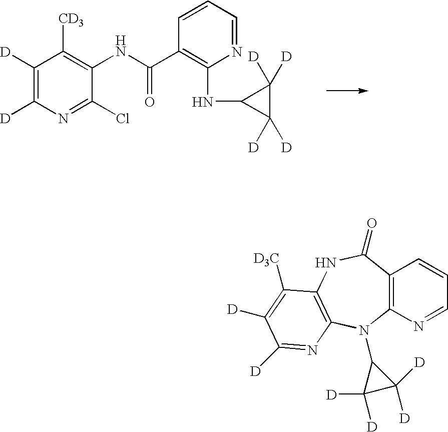

d₉-11-Cyclopropyl-5,11-dihydro-4-methyl-6H-dipyrido[3,2,-b:2′,3′-e][1,4]-diazepin-2-one (d₉-Nevirapine): The title compound is prepared according to Example 1, step 3, by substituting d₉-N-(2-chloro-4-methylpyridin-3-yl)-2-cyclopropylamino-nicotinamide for d₄-N-(2-chloro-4-methylpyridin-3-yl)-2-cyclopropylamino-nicotinamide.

### EXAMPLE 3

### d₈-11-Cyclopropyl-5,11-dihydro-4-methyl-6H-dipyrido[3,2,-b:2′,3′-e][1,4]-diazepin-2-one (d₈-Nevirapine)

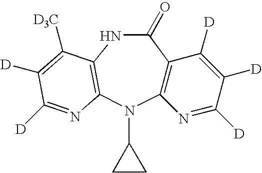

### Step 1

Activated cuprous iodine: Cuprous iodide (10 g) and sodium bisulfate (1 mol/L, 50 ml) were placed in a round-bottom flask. Sulfuric acid (1 mol/L, 10) was added and the solution was stirred for 15 minutes at ambient temperature. The solution was filtered; the filter cake was washed with water (50 mL) and tetrahydrofuran (50 mL×3), and then dried under reduced pressure.

### Step 2

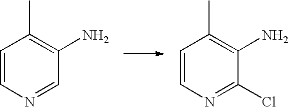

2-chloro-4-methyl-pyridin-3-ylamine: A solution of 4-methylpyridin-3-amine (200 g, 1.85 mol, 1.00 equiv) in concentrated hydrochloric acid (3 L) was placed in a 5 L 3-necked round-bottom flask. Hydrogen peroxide (30%) (210 g, 1.85 mol, 1.00 equiv) was added dropwise to the solution while maintaining the temperature at 20° C. The resulting solution was allowed to react overnight at ambient temperature. Saturated aqueous sodium carbonate was added to the solution, till a pH of 8 was reached. The solution was filtered, and the filter cake was washed with water (100 ml×3). The filter cake was dissolved in ethyl acetate (300 mL) and dried over sodium sulfate. The solution was filtered, and the filtrate was concentrated in vacuo using a rotary evaporator. The product (204.6 g, purity: 90%, yield: 78%) of 2-chloro-4-methyl-pyridin-3-ylamine was obtained as a light red solid. The material was used in next step without further purification.

### Step 3

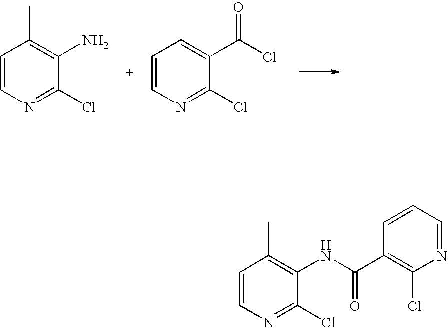

2-Chloro-N-(2-chloro-4-methyl-pyridin-3-yl)-nicotinamide: Pyridine (125 g, 1.58 mol, 1.10 equiv) was added to a solution of 2-chloro-4-methyl-pyridin-3-ylamine (204.6 g, 1.44 mol, 1.00 equiv) in acetonitrile (1500 ml) in a 2 liter 3-necked round-bottom flask. 2-chloronicotinoyl chloride (270 g, 1.54 mol, 1.07 equiv) was added dropwise to the solution while maintaining the temperature at 20° C. The solution was allowed to react overnight while maintaining the temperature at 45° C. in an oil bath. The solution was then diluted with water (2 L) and sodium carbonate was added till the pH of the solution reached 8. The solution was filtered, the filter cake was washed with water (100 mL×3), and the filter cake was dissolved in tetrahydrofuran (3 L). The solution was decolorized by the addition of active carbon, and then filtered. The filtrate was then dried over sodium sulfate, concentrated in vacuo using a rotary evaporator. The product of 2-chloro-N-(2-chloro-4-methyl-pyridin-3-yl) nicotinamide (320 g, purity: 94%, yield: 79%) was obtained as a light red solid. The material was used in next step without further purification.

### Step 4

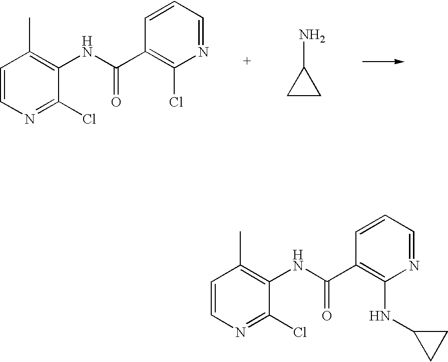

N-(2-Chloro-4-methyl-pyridin-3-yl)-2-cyclopropylamino-nicotinamide: Cyclopropanamine (120 g, 2.11 mol, 10.00 equiv) and calcium oxide (24 g, 428.57 mmol, 2.00 equiv) were added to a solution of 2-chloro-N-(2-chloro-4-methyl-pyridin-3-yl)nicotinamide (60 g, 213.52 mmol, 1.00 equiv) in xylene (300 ml) in a 1 L high pressure reactor. The solution was allowed to react overnight while maintaining the temperature at 140° C. The solution was filtered, the filter cake was washed with tetrahydrofuran (50 mL×2), and the filtrate was concentrated in vacuo using a rotary evaporator. The residue was purified by flash chromatography on silica gel (10% ethyl acetate in petroleum ether) The product of N-(2-chloro-4-methyl-pyridin-3-yl)-2-cyclopropylamino-nicotinamide (51.1 g, purity: 95%, yield: 79%) was obtained as a light yellow solid.

### Step 5

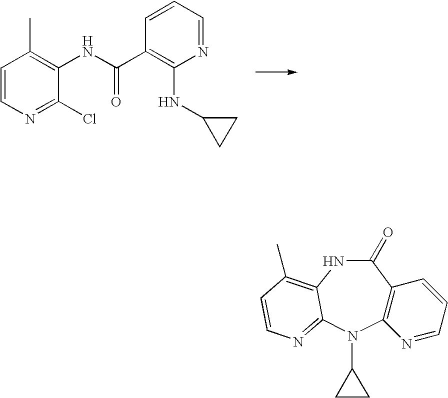

11-Cyclopropyl-5,11-dihydro-4-methyl-6H-dipyrido[3,2,-b:2′,3′-e][1,4]-diazepin-2-one (Nevirapine): A solution of N-(2-chloro-4-methylpyridin-3-yl)-2-(cyclopropylamino)nicotinamide (10 g, 33.11 mmol, 1.00 equiv) in 1-methoxy-2-(2-methoxyethoxy)ethane (300 ml) was placed in a 500 ml 3-necked round-bottom flask. The reaction was purged with nitrogen and maintained under an inert atmosphere of nitrogen. Potassium 2-methylpropan-2-olate (11 g, 98 mmol, 3.00 equiv) and activated cuprous iodide (5 g, 26 mmol) were added to the solution. The solution was allowed to react overnight while maintaining the temperature at 115° C. in an oil bath. The solution was filtered, the filter cake was washed with ethyl acetate (50 mL), and the filtrate was collected and concentrated in vacuo using a rotary evaporator. The residue was purified by flash chromatography on silica gel (10% ethyl acetate in petroleum ether). The final product (6.3 g, purity: 98%, yield: 71%) was obtained as a yellow solid.

### Step 6

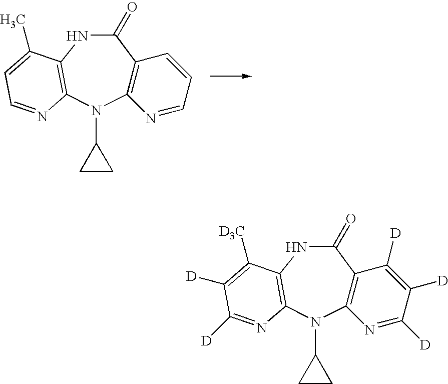

d₈-11-Cyclopropyl-5,11′-dihydro-4-methyl-6H-dipyrido[3,2,-b:2′,3′-e][1,4]-diazepin-2-one (d₈-Nevirapine): A mixture of 11-cyclopropyl-5,11-dihydro-4-methyl-6H-dipyrido[3,2,-b:2′,3′-e][1,4]-diazepin-2-one (nevirapine) (500 mg, 1.88 mmol), 10% Pd/C (20% weight, 0.1 g), sodium formate (64 mg, 0.98 mmol), deuterium oxide and dioxane (11 ml, D₂O/dioxane, 10/1, v/v) was degassed by bubbling a stream of nitrogen into the mixture for 2 minutes. The reaction was then heated to 200° C. for 48 hours. The reaction was cooled, filtered, washed with methanol, concentrated, and purified by flash column to give a white solid (299 mg, 58% yield). This solid was then further purified by preparative HPLC (methanol/water, 40/60, v/v, isogradient) to afford 100 mg (pure) of the title compound. ¹H NMR (300 MHz, CDCl₃) δ 8.55 (s, 0.02H), 8.16 (s, 0.09H), 8.13 (s, 0.96H), 7.64 (br, 1H), 7.07 (d, 0.19H), 6.93 (s, 0.68H), 3.77 (m, 1H), 2.33 (s, 0.13H), 1.01 (m, 2H), 0.46 (m, 2H); ESI-MS m/z=273.1, (MH⁺); HPLC, 99.481% (214 nm), 97.292 (254 nm).

Changes in the metabolic properties of the compounds in Examples 1 to 3 as compared to their non-isotopically enriched analogs can be shown using the following assays. Other compounds listed above, which have not yet been made and/or tested, are predicted to have changed metabolic properties as shown by one or more of these assays as well.

### Biological Assays

### EXAMPLE 4

### In Vitro Metabolism Using Human Cytochrome P₄₅ Enzymes

The cytochrome P450 enzymes are expressed from the corresponding human cDNA using a baculovirus expression system (BD Biosciences). A 0.25 milliliter reaction mixture containing 0.8 milligrams per milliliter protein, 1.3 millimolar NADP+, 3.3 millimolar glucose-6-phosphate, 0.4 U/mL glucose-6-phosphate dehydrogenase, 3.3 millimolar magnesium chloride and 0.2 millimolar of a compound of Formula 1, the corresponding non-isotopically enriched compound or standard or control in 100 millimolar potassium phosphate (pH 7.4) is incubated at 37° C. for 20 min. After incubation, the reaction is stopped by the addition of an appropriate solvent (e.g. acetonitrile, 20% trichloroacetic acid, 94% acetonitrile/6% glacial acetic acid, 70% perchloric acid, 94% acetonitrile/6% glacial acetic acid) and centrifuged (10,000 g) for 3 minutes. The supernatant is analyzed by HPLC/MS/MS.

| Cytochrome P₄₅₀ | Standard |
|---|---|
| CYP1A2 | Phenacetin |
| CYP2A6 | Coumarin |
| CYP2B6 | [¹³C]-(S)-mephenytoin |
| CYP2C8 | Paclitaxel |
| CYP2C9 | Diclofenac |
| CYP2C19 | [¹³C]-(S)-mephenytoin |
| CYP2D6 | (+/−)-Bufuralol |
| CYP2E1 | Chlorzoxazone |
| CYP3A4 | Testosterone |
| CYP4A | [¹³C]-Lauric acid |

### EXAMPLE 5

### Monoamine Oxidase A Inhibition and Oxidative Turnover

The procedure is carried out as described in Weyler, Journal of Biological Chemistry 1985, 260(24), 13199-13207, which is hereby incorporated by reference in its entirety. Monoamine oxidase A activity is measured spectrophotometrically by monitoring the increase in absorbance at 314 nm on oxidation of kynuramine with formation of 4-hydroxyquinoline. The measurements are carried out, at 30° C., in 50 mM NaPᵢ buffer, pH 7.2, containing 0.2% Triton X-100 (monoamine oxidase assay buffer), plus 1 mM kynuramine, and the desired amount of enzyme in 1 mL total volume.

### EXAMPLE 6

### Monoamine Oxidase B Inhibition and Oxidative Turnover

The procedure is carried out as described in Uebelhack, Pharmacopsychiatry 1998, 31(5), 187-192, which is hereby incorporated by reference in its entirety.

### EXAMPLE 7

### MAO Assay

Fresh PRP or frozen platelet suspension (100 μl) is generally preincubated for 10 minutes in the absence or presence of drugs at 37° C. in 100 μl of 0.9% NaCl solution or phosphate buffer pH 7.4, respectively, at 37° C. 2-Phenyllethylamine-[ethyl-1-¹⁴C]hydrochloride (PEA) solution (specific activity 56 Ci/mol, Amersham, 50 μl) is then added to reach a final concentration of 5 μM and incubation is continued for 30 minutes. The reaction is terminated by the addition of 50 μl 4M HClO₄. The reaction product of MAO, phenylacetaldehyde, is extracted into 2 mL of n-hexane. An aliquot of the organic phase is added to scintillator cocktail and the radioactivity is determined using a liquid scintillation counter. Product formation is linear with time for at least 60 min with appropriate platelet numbers. Blank values are obtained by including 2 mM pargyline in the incubation mixtures.

### EXAMPLE 8

### Preparation of Platelet-Rich Plasma and Platelets

Venous blood from healthy subjects is collected between 8 and 8:30 a.m. after overnight fasting into EDTA-containing vacutainer tubes (11.6 mg EDTA/mL blood). After centrifugation of the blood at 250×g for 15 minutes at 20° C., the supernatant platelet-rich plasma (PRP) is collected and the number of platelets in PRP are counted with a cell counter (MÖLAB, Hilden, Germany). PRP (2 mL) is spun at 1500×g for 10 minutes to yield a platelet pellet. The pellet is washed three times with ice-cold saline, resuspended in 2 mL Soerensen phosphate buffer, pH 7.4 and stored at −18° C. for one day.

### EXAMPLE 9

### In vitro Liver Microsomal Stability Assay

Liver microsomal stability assays are conducted at 1 mg per mL liver microsome protein with an NADPH-generating system in 2% NaHCO₃ (2.2 mM NADPH, 25.6 mM glucose 6-phosphate, 6 units per mL glucose 6-phosphate dehydrogenase and 3.3 mM MgCl₂). Test compounds are prepared as solutions in 20% acetonitrile-water and added to the assay mixture (final assay concentration 5 microgram per mL) and incubated at 37° C. Final concentration of acetonitrile in the assay should be <1%. Aliquots (50 μL) are taken out at times 0, 15, 30, 45, and 60 minutes, and diluted with ice cold acetonitrile (200 μL) to stop the reactions. Samples are centrifuged at 12000 RPM for 10 minutes to precipitate proteins. Supernatants are transferred to microcentrifuge tubes and stored for LC/MS/MS analysis of the degradation half-life of the test compounds.

### EXAMPLE 10

### In Vitro Antiviral Assays

The procedure is carried out as described in Hazen, Antimicrobial Agents and Chemotherapy 2005, 49(11), 4465-4473, which is hereby incorporated by reference in its entirety.

### Construction of Isogenic HIV-1 Mutant Virus for Passage and Virus Sensitivity Testing:

A panel of isogenic viruses that possess mutations of interest in the RT-coding region is prepared from HIV-1 strain HXB2. Specific amino acid substitutions in the HXB2 RT are generated by site-directed mutagenesis of the RT DNA carried by plasmid pRT2. The codon changes, which yield K103N, Y181C, V106A, V106I, P236L, and V106I-P236L mutations, are verified by nucleoside sequence determination of the entire RT-coding region on both DNA strands. Isogenic recombinant viruses are recovered following cotransfection of MT-4 cells with linearized, mutant RT plasmids and molecular clone HXB2 RTBstII from which RT is deleted. The recombinant progeny virus is expanded in MT-4 cells and harvested, the titer is determined, and the sequence is verified.

### MT-4 Cell Assay.

Anti-HIV activity and compound-induced cytotoxicity are measured in parallel by means of a 3-(4,5-dimethylthiazol-2-yl)-5-(3-carboxymethoxyphenyl)-2-(4-sulfophenyl)-2H-tetrazolium, inner salt (MTS)-based procedure in MT-4 cells. Aliquots of the test compounds are serially diluted in medium (RPMI 1640, 10% [vol/vol] FBS, and 10 μg/ml gentamicin) in 96-well plates. Exponentially growing MT-4 cells are harvested and centrifuged at 192×g for 10 minutes in a Jouan centrifuge. Cell pellets are re-suspended in fresh medium (RPMI 1640, 20% [vol/vol] FBS, 20% [vol/vol] interleukin-2 [IL-2], and 10 μg/ml gentamicin) to a density of 5×10⁵ cells/ml. Cell aliquots are infected by the addition of HIV-1IIIB diluted to give a viral inoculum of one hundred 50% tissue culture infective doses (TCID₅₀s) per well. A similar cell aliquot is diluted with medium to provide a mock-infected control. Cell infection is allowed to proceed for 1 hour at 37° C. in a tissue culture incubator with a humidified 5% CO₂ atmosphere. After incubation, the virus-treated cell suspensions are diluted six fold with fresh medium, and 125 μl of the cell suspension is added to each well of the plate containing pre-diluted compound. The test compound is tested over final concentration ranges of 0.5 to 500 nM. The plates are then placed in a tissue culture incubator at 37° C. with humidified 5% CO₂ for 5 days. HIV-induced cytopathic effects are assessed by a CellTiter 96 MTS staining method (catalog no. G3581; Promega, Madison, Wis.). The optical density at 492 nm is measured by using a microplate absorbance reader (catalog no. 20-300; Tecan, Research Triangle Park, N.C.).

### Combination Antiviral Activity Assay in MT-4 Cells

Anti-HIV activity and compound-induced cytotoxicity are measured in parallel in an MT-4 cell assay. Aliquots of the test compound are serially diluted vertically in a 96-well master assay plate, in medium (RPMI 1640, 10% [vol/vol] FBS, and 10 μg/ml gentamicin), at concentrations that are 40-fold higher than the final assay concentration. Approved HIV inhibitors are diluted horizontally across master assay plates, also at concentrations that are 40-fold higher than the final assay concentration. Checkerboard-style dilutions are arranged so that every concentration of the test compound is tested in the presence and the absence of every concentration of the approved HIV inhibitor.

### PBMC Assay

PHA- and IL-2-stimulated PBMCs are pelleted at 192×g for 10 minutes in a Jouan centrifuge and re-suspended to 4×10⁶ cells/ml in RPMI 1640, 20% (vol/vol) FBS, 10% (vol/vol) IL-2, and 10 μg/ml gentamicin; and 100 μl is distributed to 96-well tissue culture plates. Test compounds are titrated four fold into RPMI 1640 20% (vol/vol) FBS, 10% (vol/vol) IL-2, and 10 μg/ml gentamicin at four times the final assay concentration. Fifty microliters of titrated inhibitor is dispensed onto the 100 μl of PBMCs and incubated at 37° C. in 5% CO₂ for 1 hour. Fifty microliters of diluted HIV-1IIIB is then added to each well, and the contents of the plates are thoroughly mixed. The test compound is assayed over a final concentration range of 0.003 to 5,000.00 nM. The plates are placed in a humidified incubator at 37° C. with 5% CO₂ for 7 days. On day 7 of the assay, 50 μl of the culture supernatants is transferred to a new 96-well plate. The RT levels in the supernatants are measured.

Series One Passage with Wt HIV-1HXB2

Selection for resistant variants is performed by sequential passage of HIV-1 strain HXB2 in escalating concentrations of test compounds. For the initial passage, the test compound is present at approximately the IC₅₀ in MT-4 cells. A total of 4×10⁷ MT-4 cells is re-suspended in 500 μl of cell culture medium containing HIV-1_{HXB2} (100 TCID₅₀s per culture). Following 1 hour of virus adsorption, the virus-cell suspension is brought to a final volume of 24 mL by the addition of RPMI 1640 medium containing 10% (vol/vol) FBS (1.6×10⁶ MT-4 cells/ml). Five hundred microliters of the infected cell suspension is added per well in a 48-well tissue culture plate containing 500 μl of diluted test compound. Six parallel lineages are prepared for each test compound. Cultures are incubated at 37° C. in a 5% CO₂ humid atmosphere. Samples of the cell supernatant are collected from each culture at 2- to 4-day intervals and monitored for the levels of RT. Cultures containing approximately 125,000 RT cpm/30 μl or more are harvested. After centrifugation at 192×g for 10 minutes in a Jouan centrifuge, the supernatants are collected for further passage, titer determination, nucleotide sequencing, and sensitivity testing. For the subsequent passages, fresh MT-4 cells (4×10⁵) are infected with 300 μl of culture medium containing virus from the previous passage (regardless of the virus titer) and cultured in the presence of the compound at concentrations that are increased two-fold compared with that in the previous passage. This procedure is repeated with increasing concentrations of the test compound for eight passages.

Series Two Passage with HXB2 Containing WT or Site-Directed RT Mutations Conferring NNRTI Resistance.

Selection for drug-resistant HIV variants is performed by sequential passage of HIV-1_{HXB2} strains in escalating concentrations of the test compound. The WT and strains containing site-directed RT mutations associated with NNRTI resistance are used as the starting virus. The selection of mutations in the starting virus is based on key mutations that confer resistance to current NNRTIs (e.g., K103N, Y181C, and V106A) or, where identified, as mutations that are selected by passage in the presence of the test compound in the initial passage series (e.g., V106I, P236L, and V106I-P236L). For the first passage, either 0.5 or 1 nM compound is chosen as the starting concentration, based on the sensitivity of the non-passaged mutant viruses to the test compound in a HeLa MAGI (multinuclear activation of galactosidase indicator) assay system. A total of 2×10⁶ MT-4 cells are re-suspended in 100 μl of culture medium containing HIV-1 (titrated to result in the addition of 100 TCID₅₀s per culture). The virus suspension is brought to a final volume of 10 mL by the addition of RPMI 1640 medium containing 10% (vol/vol) FBS with or without compound. The cultures are incubated in 25-cm² tissue culture flasks at 37° C. in a 5% CO₂ humid atmosphere. Samples of the cell supernatant are collected from each culture at 2- to 4-day intervals and monitored for the levels of RT. Cultures containing approximately 125,000 RT cpm/30 μl or more are harvested. After centrifugation at 192×g for 10 minutes in a Jouan centrifuge, the supernatants are collected for further passage, titer determination, nucleotide sequencing, and sensitivity testing. If the cultures do not contain RT levels high enough for harvest, fresh medium and compound are added and incubation is continued. For the subsequent passages, fresh MT-4 cells are infected with 100 μl of culture medium containing virus (regardless of the virus titer) and cultured in the presence of GW678248, typically at twofold increased concentrations. Exceptions are made when virus breakthrough failure lead to the restart of a passage at the same or a lower concentration than the previous passage.

### Virus Infectivity Titration in MT-4 Cells

The infectivity titers of serially passaged virus are determined in MT-4 cells. Seven serial four-fold dilutions of passage supernatants containing virus, ranging from 1:16 through 1:65,536, in a volume of 125 μl are titrated in triplicate in 96-well culture plates. Uninfected MT-4 cells are pelleted at 192×g for 10 minutes in a Jouan centrifuge and re-suspended in RPMI 1640 medium with 15% (vol/vol) FBS and 10% (vol/vol) IL-2 at a concentration of 8.3×10⁶ cells/mL. A 125-μl aliquot of cell suspension is added to the wells, which contained diluted virus. The plates are incubated for 5 days at 37° C. in a humid 5% CO₂ atmosphere. HIV-induced cytopathic effects are assessed by an MTS staining method. The numbers of TCID₅₀ per mL for each virus are determined by the Spearman-Karber method.

### Drug Sensitivity Determination

The drug sensitivities of HXB2 and virus passaged in the presence of the test compound are determined in a modified MT-4 cell assay. MT-4 cells (1×10⁶ per incubation) are incubated with 300 TCID₅₀s of test HIV for 1 hour at 37° C. Following incubation, the virus-cell suspension is diluted 10-fold with RPMI 1640 medium with 15% (vol/vol) FBS and 10% (vol/vol) IL-2. Subsequently, 125 μl of the virus-cell suspension is plated per well of a 96-well microtiter plate containing 125 μl of serially diluted test compound. The compounds are serially diluted 1:3.16 in the series two experiments. The plates are incubated for 5 days at 37° C. HIV-induced cytopathic effects are assessed by an MTS staining method.

### Genotype Analysis of Passaged Virus

The DNA sequences of the entire protease (PR)-coding region (codons 1 to 99) and the first 235 codons of the RT-coding region are determined from passaged virus supernatants. Viral RNA is extracted from cell-free supernatants by lysis in guanidinium isothiocyanate, followed by alcohol precipitation. Target cDNA is generated by RT-PCR with the ViroSeq HIV-1 genotyping system (Celera Diagnostics, Alameda, Calif.). Sequencing primers used for the RT-coding region are 5′-AATTTTCCCATTAGTCCTATTGAAACTGTACCAG (SEQ ID NO: 1) and 5-CCCCACTAACTT CTGTATGTCATTGAC (SEQ ID NO: 2); the primers used for the PR coding region are 5′-G CCGATAGACAAGGAACTGTATCC (SEQ ID NO: 3) and 5-TGAAAAATATGCATCACC (SEQ ID NO: 4).

### EXAMPLE 11

### In Vivo Pharmacology

### Incidence of Skin Rash in BN Rats

The procedure is carried out as described in Shenton, Chem. Res. Toxicol. 2005, 18, 1799-1813, which is hereby incorporated by reference in its entirety. Rats (150-175 g) are housed in pairs in standard cages in a 12:12 hour light/dark cycle at 22° C. with free access to water and chow. After a 1-week acclimation period during which food intake is monitored, the rats are either continued on the chow diet or switched to a diet containing test compound (150 mg/kg per day). Animals are monitored for development of skin rash, food intake, and body weight. At the endpoint of the experiment, rats are sacrificed by i.p. injection (3 mL/kg) of an anesthetic mixture (5:3 ketamine (100 mg/mL) to xylazine (20 mg/mL)). Experiments involving re-challenge (i.e., secondary exposure to test compound) refer to rats that are treated with test compound until skin rash developed, then removed from drug for 4 weeks (unless otherwise stated), and finally re-exposed to test compound. For re-challenge experiments, the rats are placed on the same test compound-containing diet as for primary exposure.

### Treatments to Modify the Incidence of Skin Rash

1. Pre-Treatment with Poly(I:C) or Misoprostol.

For poly(I:C) pre-treatment, rats (n=8) are treated with a single dose of poly(I:C) (10 mg/kg, i.p., 1 day prior to initiation of test compound treatment); the poly(I:C) is dissolved over several hours (rather than to use vigorous agitation which might mechanically rupture the molecules) in PBS. Control groups include rats (n=3) that are treated with poly(I:C), but not started on test compound, and rats (n=2) treated with test compound only. For misoprostol pre-treatment, rats (n=4) are treated with a single dose of misoprostol (300 μg/kg sc) in corn oil 1 day prior to initiation of test compound treatment. The control rats (n=2) are treated with corn oil only.

2. Co-Treatment with Aminoguanidine.

Rats (n=2) are treated with a single dose of aminoguanidine (100 mg/kg, i.p.) in sterile saline. The following day, the rats are started on test compound and drinking water supplemented with 0.1% (w/v) aminoguanidine resulting in a daily dose of approximately 100 (mg/kg)/day. Control rats (n=2) are treated with aminoguanidine only.

3. Co-Treatment with Cromolyn, Ketanserin, and Astemizole.

Rats (n=4) are treated with cromolyn (300 (mg/kg)/day i.p.; prepared in sterile water), astemizole (5 (mg/kg)/day by gavage; prepared in a 0.5% methyl cellulose suspension in water), and ketanserin (0.1 (mg/kg)/day s.c.; prepared in sterile PBS) starting 1 day prior to initiation of test compound treatment. Treatment with test compound and cromolyn, ketanserin, and astemizole is continued for 14 days. The control groups include rats (n=2) that are treated with test compound only and rats (n=2) that are treated with cromolyn, ketanserin, and astemizole only. The cromolyn and astemizole solutions are prepared weekly, while the ketanserin solution is prepared daily.

4. Co-Treatment with the Immunosuppressant Tacrolimus or Cyclosporine.

Rats are treated with tacrolimus (1 (mg/kg)/day dissolved in PBS; for initial experiments, tacrolimus is administered i.m., but chronic i.m. injections are stressful for the animals, so the route of exposure is switched to s.c.) or cyclosporine (20 (mg/kg)/day s.c. dissolved in olive oil).

Treatment of Skin Rash with Tacrolimus

In naive animals, tacrolimus treatment is initiated on day 7 of test compound treatment (i.e., once the rats presented with red ears). To determine if tacrolimus treatment must be continued indefinitely to prevent rash, tacrolimus treatment is stopped in some animals. To determine if tacrolimus could reduce recovery time after severe rash, test compound treatment is discontinued on day 9 of re-challenge, and daily treatment with tacrolimus (1 (mg/kg)/day; starting 10 hour after test compound treatment is discontinued) is initiated.

### Experiments to Determine if Tolerance Induced by Low-Dose Treatment (Low-Dose Tolerance) is Metabolism- or Immune-Mediated

### 1. Determination of Whether the Observed Tolerance has Memory.

Rats are administered a low dose of test compound (40 (mg/kg)/day) for 2 weeks and then removed from drug for 1 week (n=2) or 4 weeks (n=4). At the end of the 1-week or 4-weeks no-treatment period, the low-dose pretreated rats and naive control rats (n=2) are started on test compound (150 (mg/kg)/day). If tolerance is mediated by induction of metabolism, it should not have long-term memory.

### 2. Adoptive Transfer of Tolerance.

Donor rats (n=2) are administered a low dose of test compound (40 (mg/kg)/day) for 2 weeks and then transferred to the full dose. Spleen cell suspensions are prepared and pooled from the donor rats and injected i.v. via the tail vein into naive recipients (n=2). Thus, each recipient rat receives roughly the entire complement of splenocytes from the spleen of a donor rat. The following day, the recipient animals are started on test compound. In a second experiment, splenocytes are isolated from a single donor rat on the final day of low-dose test compound treatment (day 14). The splenocytes are injected into a single naive recipient, and 2 hour later, the naive recipient is started on test compound. In the experiment, a single naive recipient also receives splenocytes from a naive donor (to serve as a control) prior to test compound dosing. Spleen cell suspensions are prepared as described below with the following changes: the cells are washed once in PBS (following red cell lysis) before re-suspension in PBS in an appropriate volume for i.v. injection (the cells are not counted). If tolerance is immune mediated, then it may be possible to transfer tolerance with splenocytes from a tolerant donor to a naive recipient.

3. Inhibition of P450-Mediated Metabolism by Co-Treatment with ABT.

Rats (n=4) are treated with a low dose of test compound (40 (mg/kg)/day) for 2 weeks. After 2 weeks of low-dose treatment, the rats are switched to the full dose of test compound but also begin treatment with ABT (20 (mg/kg)/day in water by gavage). Test compound plasma levels are monitored, and the ABT dose are increased if the levels drop below the values recorded previously in the rats that are treated with test compound (150 (mg/kg)/day, i.e., the full dose) from the onset of treatment. If tolerance is mediated by induction of metabolism, then inhibiting metabolism after low-dose treatment should reverse the low-dose tolerance.

### Experiments to Determine if Tolerance Induced by Tacrolimus Co-Treatment is Metabolism- or Immune-Mediated.

Female BN rats (n=6) are co-treated with test compound and tacrolimus (0.7 (mg/kg)/day in PBS s.c.) for 5 weeks. Rats (n=2) are treated with test compound alone to serve as a control group. After 5 weeks of co-treatment, the rats are treated with test compound only. Three weeks later, ABT dosing is added (20 (mg/kg)/day in tap water; gavage) to increase the test compound plasma concentration to rash-inducing levels. Test compound plasma levels are monitored, and the ABT dose are increased if the levels drop below the values recorded previously the rats that are treated with test compound (150 (mg/kg)/day, i.e., the full dose) from the onset of treatment. Two out of four rats are continued on test compound alone to serve as controls. After 5 weeks, all treatments are discontinued (n=6 rats). If tolerance is mediated by induction of metabolism, then inhibiting test compound metabolism after tacrolimus treatment should reverse the tolerance. Furthermore, if tolerance is mediated by induction of metabolism, then it should not have memory.

### Preparation of Spleen, Peripheral Blood, and Lymph Node Cell Suspensions

Spleens and lymph nodes are collected and processed in cold RPMI 1640 (flow cytometry studies) or PBS (adoptive transfer studies) supplemented with 5% FBS. Spleens are crushed using the butt end of a sterile 10 cm³ syringe plunger to release the cells. Cell suspensions are passed through a 70 μm Falcon nylon mesh cell strainer (Becton Dickinson, Franklin Lakes, N.J.), centrifuged, and then re-suspended in 10 mL of ammonium chloride buffer (155 mM NH₄Cl, 10 mM KHCO₃, and 0.1 mM EDTA) for 10 minutes to lyse red blood cells. The cells are washed once, re-suspended in PBS, and counted (viability assessed in 0.4% trypan blue solution). Mesenteric and popliteal lymph nodes are processed in the same manner as the spleens, except a smaller plunger is used (5 cm³ syringe) and the red cell lysis step is omitted. Peripheral blood is drawn from the abdominal aorta and/or heart into a lavender-top Vacutainer (containing EDTA anticoagulant; Becton Dickinson). Peripheral blood mononuclear cells are isolated using Lympholyte-Mammal (CedarLane) following the manufacturer's instructions. Isolated peripheral blood mononuclear cells are washed once in PBS and then counted as for splenocytes. Centrifugation steps are carried out at 340 g, 6 minutes, and 4° C.

### Adoptive Transfer of Susceptibility to Skin Rash

The subpopulations are injected i.v. via the tail vein into naive recipients. The naive recipients are placed on test compound ≦2 hour after the i.v. injections. Subsets of splenocytes are obtained using enrichment columns (R&D Systems; Minneapolis, Minn.). A test experiment is completed on each type of enrichment column in which the final elutes are checked by flow cytometry to verify the enrichment of the respective cell type. T cells are stained with antibodies against both CD3 and αβ T cell receptor (TCR); CD4⁺ T cells and CD8⁺ T cells are identified by double staining with antibodies against CD4 and αβ TCR or CD8β and αβ TCR, respectively; CD11b/c or CD45RA positive cells are considered to be macrophages or B cells, respectively; and finally, cells positive for CD8α are reported.

In order to determine the ability of splenocytes from rats on day 21 of primary exposure to transfer susceptibility, total splenocytes are isolated from donor rats on day 21 of primary test compound treatment (n=2) or day 9 of re-challenge (n=10), suspended in PBS, and injected i.v. via the tail vein into naive recipients, n=2 or n=10, respectively. Thus, each recipient rat receives approximately the entire complement of splenocytes from the spleen of a donor rat. The naive recipients are placed on test compound either ≦2 hour after or the day following the i.v. injections.

The ability of lymph node cells, instead of splenocytes, to adoptively transfer susceptibility can be evaluated. “Total” lymph node cells (pooled popliteal and mesenteric) are isolated from donor rats (n=8) on day 9 of re-challenge and injected i.v. via the tail vein into n=5 recipients. The recipients receive varying amounts of lymph node cells, 100×10⁶ cells, 50×10⁶ cells, 30×10⁶ cells, and all the cells from one donor animal. The naive recipients are placed on test compound 2 hours following the i.v. injections.

The capacity of the humoral arm of the immune system to adoptively transfer susceptibility is assayed. Plasma is isolated from donor rats (n=4) on day 9 of re-challenge. Age-matched untreated rats (n=2) serve as control donors. Blood is isolated from the abdominal aorta and/or the heart into a lavender-top Vacutainer. Blood is centrifuged at 600 g, 4° C., for 20 min, and the plasma from treated rats or control rats is pooled separately. Recipient rats are injected i.v. with approximately 2.7 mL of treated donor plasma (n=4) or 0.8 mL of control donor plasma (n=2) via the tail vein. The following day, the control plasma recipients (n=2) and treated plasma recipients (n=2) are started on test compound, whereas the remaining (n=2) treated plasma recipients are administered the control diet.

Adoptive transfer is considered to have transferred susceptibility if the naive recipient developed red ears <24 hour after starting test compound, that is, similar to a previously sensitized animal re-challenged with nevirapine.

### CD4⁺ or CD8⁺ T Cell Depletion

Thymectomized female BN rats are cared for as described above with the exception that housing, water, and chow are kept sterile. For rats to be depleted of CD8⁺ T cells, rats (n=6) are injected i.p. with anti-CD8α antibody 10 days (1.3 mg) and 6 days (0.5 mg) prior to the start of test compound treatment. Three days prior to the start of test compound treatment, a rat is sacrificed to confirm CD8⁺ T cell depletion by flow cytometry. Thymectomized rats (n=2) treated with an isotype control antibody (IgG1) in pilot studies had normal levels of CD8⁺ T cells. Just prior to initiation of test compound treatment, another 0.65 mg of anti-CD8α antibody is administered to each rat to ensure the continued absence of CD8⁺ T cells. For rats to be depleted of CD4⁺ T cells, rats (n=6) are injected i.p. with anti-CD4 antibody (7 mg/kg) for 5 consecutive days. Twenty-four hour after the last dose, rats (n=2) are sacrificed to determine the degree of CD4⁺ T cell depletion using flow cytometry; Thymectomized rats (n=2) that are treated with an isotype control antibody (IgG2a) serve as controls for the CD4⁺ T cell depletion experiment. At the termination of the experiment, flow cytometry analysis is again conducted on spleen and lymph node cell suspensions (CD8⁺ T cell depletion) as well as peripheral blood mononuclear cells (CD4⁺ T cell depletion) to verify depletion.

### Analysis of T Cell Subpopulations in the Spleen and Lymph Nodes of Treated Rats

Rats are divided into three groups: control, primary, and re-challenge, with 5 rats per group. Rats in the primary treatment group are administered test compound for 21 days (or the time point when all rats have rash). For the re-challenge treatment group, rats are administered test compound for 21 days (as for the primary treatment group) and then given a 4-week break. After the 4-week break, the rats are re-exposed to test compound. Animals in the control group receive no treatment with test compound. All animals are agematched, and the experiment is timed such that rats in each treatment group can be sacrificed on the same day. At the termination of the experiment, spleen and lymph nodes (pooled popliteal and mesenteric) cell suspensions are prepared from each rat for phenotypic analysis by flow cytometry. The CD4/CD8 T cell ratio is determined by dividing the percent CD4 and αβ TCR double-stained cells by the percent CD8β and αβ TCR double-stained cells.

### Flow Cytometry

Single-cell suspensions are surfacelabeled, and one- or two-color immunofluorescence analysis is conducted. Briefly, single-cell suspensions are re-suspended in PBS. Monoclonal antibodies or suitable isotype controls are aliquoted to the appropriate wells of an untreated 96-well conical v-bottom microplate (Evergreen Scientific, Los Angeles, Calif.). The volume of antibody solution in each well is adjusted to 150 μL with PBS. Finally, 50 μL (1×10⁶ cells) of the appropriate cell suspension is aliquoted to each well. Cells are incubated with antibody solutions at room temperature in the dark for 15 min. The cells are washed twice with PBS and finally re-suspended in 1% paraformaldehyde (pH 7.4) in PBS and stored at 4° C. in the dark until analysis on a FACSCalibur (Becton Dickinson) using the CellQuest software (Becton Dickinson). During the analysis, the size of the lymph node cells in the populations identified as CD8⁺ T cells and CD4⁺ T cells is compared across treatment groups.

### Determination of Serum IgE Concentration

Weekly blood samples (<400 μL) commencing on day 0, the first day of test compound dosing, are taken via the tail vein of test compound treated rats (n=4). On day 21, the final day of blood collection, test compound treatment is interrupted for a period of 4 weeks. Subsequently, test compound treatment is restarted (n=7). Serum is obtained from the rats on secondary test compound exposure; however, blood (<200 uL) is collected on days 0 and 3 (n=3) or days-2, 0.5, and 7 (n=4). On day 9, the rats are sacrificed and blood is collected via the abdominal aorta or heart. Age-matched untreated rats (n=6) serve as controls for these experiments. Blood is centrifuged at 380 g, 4° C., for 10 minutes to isolate the serum component.

A standard calorimetric sandwich ELISA is performed to measure IgE sera concentrations; 96-well plates are blocked with blocking buffer (10% FBS in PBS) for 30 minutes at room temperature; the standard curve consists of duplicate 100 μL samples of serially 1.5-fold-diluted (125-11 ng/mL) rat IgE kappa myeloma (Serotec) in blocking buffer; serum samples are diluted at minimum 80-fold and at maximum 1440-fold in blocking buffer; standards and samples are incubated overnight at 4° C.; and the absorbance is recorded at 450 nm using a SPECTRAmax spectrofluorometer microplate reader (Molecular Devices, Sunnyvale, Calif.) after a 10 minutes reaction period with o-phenylenediamine dihydrochloride (OPD) substrate (Sigma FAST OPD tablet set; Sigma). Results are expressed in micrograms per milliliter (μg/mL) as calculated from the standard curve.

### EXAMPLE 12

### In Vivo Pharmacology

### Incidence of Skin Rash in BN Rats

The procedure is carried out as described in Popovic, Chem. Res. Toxicol. 2006, 19, 1205-1214, which is hereby incorporated by reference in its entirety. Female BN rats (150-175 g) are housed in pairs in standard cages with free access to water and Agribrands powdered lab chow diet (Leis Pet Distributing Inc., Wellesley, ON). After a one-week acclimation period during which food intake is monitored, the rats are either continued on the powdered lab chow diet (control) or switched to a diet containing test compound such that the approximate daily dose is 150 mg/kg. Rats are monitored for the development of red ears, skin rash, food intake, and body weight. After the appearance of red ears or prominent skin lesions, the rats are anesthetized by an i.p. injection (1-2 mL/kg) of a ketamine (100 mg/mL) and xylazine (20 mg/mL) mixture (5:3 ratio by volume) and killed by exsanguinations.

### Time Course Study.

Female BN rats (n=48) are either fed a powdered lab chow diet (n=24, four rats per time point) or a diet delivering 150 mg/kg/day of test compound (n=24, four rats per time point), until they develop red ears or skin lesions. Upon re-challenge with test compound, rats previously treated with test compound should develop red ears within 1 day and skin lesions within 9 days. Control rats are fed commercially supplied powdered chow diet on both primary and re-challenge exposures. On the basis of this data, animals are sacrificed after 7, 14, and 21 days of primary test compound exposure and 0, 1, and 9 days of re-challenge, and sera, ALNs, and ear sections are collected.

### Preparation of ALNs for Flow Cytometry Analyses

ALNs are excised, put into Petri dishes containing culture medium (50 mL of FCS, 5 mL of MEM nonessential amino acids, 5 mL of antiinfective s, 5 mL of diluted 2-ME (35 μL of 2-ME in 100 mL of distilled water) and 435 mL of 1640 RPMI-HEPES modified). ALN cells are teased out of the nodal capsule and filtered through a 70 μm mesh (VWR). Cells are spun down at 200 g, 4° C. for 6 min, re-suspended in FACS buffer (100 μL/10 mL FCS, 5 mL of sodium azide, 485 mL of PBS), and counted. One million cells are plated in each well of the 96-well V bottom polystyrene microplates (Hospital Logistics, Inc., Oakville ON) and stained at 4° C. for 15 min, first with the anti-Fcγ receptor antibody to prevent nonspecific staining, followed by the primary antibodies for various cell surface markers. In the case of biotin-labeled primary antibodies, the cells are incubated for 15 minutes with streptavidin-APC and then washed once with the FACS buffer (100 μL). Data are acquired by FACSCalibur (BD Biosciences) and analyzed using CellQuest software (BD Biosciences).

### Preparation of Ear Sections for Immunohistochemistry

Skin Patch Test. Female BN rats are fed rat chow containing test compound (150 mg/kg/day dose, n=15) until they develop skin lesions. After a period of 28 days during which skin lesions resolve, a single dose of test compound, or vehicle control is administered onto the inner side of the rat's right ear. Then, 100 μL of a vehicle (acetone/olive oil, 1:1/v:v), 2.5 mg/mL, 0.5 mg/mL of test compound are applied to the right ear of a rat (n=3). A day after applying test compound, any change in the rats' ear color is noted. Three days later, the rats are sacrificed and both right and left ears are longitudinally dissected and prepared for the immunohistochemistry analyses.

### ELISA

Blood is obtained from these rats by cardiac puncture, stored overnight at 4° C. in Vacutainer red top 10 mL tubes (VWR; Mississauga, Ontario), and centrifuged the following morning at 150 g at 4° C. for 10 minutes. Rat serum IFNγ analysis is performed following the instructions on the BD Biosciences kit.

### EXAMPLE 13

### In Vivo Pharmacology

### Incidence of Skin Rash in BN Rats

The protocol was modeled after that published previously (Shenton et al, Chem. Res. Toxicol. 2005, 18, 1799-1813, and Popovic et al, Chem. Res. Toxicol. 2006, 19, 1205-1214). After a 1-week acclimation period, the rats (n=3 per group) were either treated with the suspension vehicle (1% Tween® 20), or Nevirapine (NVP) (150 mg/kg/day) in suspension, or compounds of Formula I (dNVP) (150 mg/kg) in suspension. Animals were monitored for development of skin rash, and body weight. At the endpoint of the experiment, rats were humanely euthanized using carbon dioxide and exsanguinations.

### Time Course Study

The control group (vehicle only) and the dNVP groups behaved normally and did not develop any observable skin lesions or other indications of poor health over the entire course of the experiment. The NVP-treated group animals all displayed inactivity and a lack of responsiveness for about three hours after each dosing. The NVP group also displayed distinctly red ears, edematous eyes, purple feet, and a mottled tail at approximately the two-week point. Shortly thereafter, all NVP-treated animals displayed lesions, particularly around the dorsal area of the neck. At day 18, all animals in all three groups were euthanized as the experiment was deemed to have reached its ethical endpoint where no additional dosing was considered appropriate. Thus, under controlled conditions, NVP appeared to be substantially more toxic than compounds of Formula I.

The examples set forth above are disclosed to give a complete disclosure and description of how to make and use the claimed embodiments, and are not intended to limit the scope of what is disclosed herein. Modifications that are obvious, in the art, are intended to be within the scope of the following claims. All publications, patents, and patent applications cited in this specification are incorporated herein by reference as if each such publication, patent or patent application were specifically and individually indicated to be incorporated herein by reference. However, with respect to any similar or identical terms found in both the incorporated publications or references and those explicitly put forth or defined in this document, then those terms definitions or meanings explicitly put forth in this document shall control in all respects.

What is claimed is:

1. A compound having structural Formula I 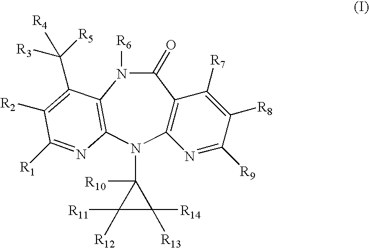
or a pharmaceutically acceptable salt, solvate, or prodrug thereof, wherein:
R₁, R₂, R₃, R₄, R₅, R₆, R₇, R₈, R₉, R₁₀, R₁₁, R₁₂, R₁₃, and R₁₄ are independently selected from the group consisting of hydrogen and deuterium;
at least one of R₁, R₂, R₃, R₄, R₅, R₆, R₇, R₈, R₉, R₁₀, R₁₁, R₁₂, R₁₃, and R₁₄ is deuterium; and
when R₃, R₄, and R₅ are deuterium then at least one of R₁, R₂, R₆, R₇, R₈, R₉, R₁₀, R₁₁, R₁₂, R₁₃, and R₁₄ is deuterium.

2. The compound as recited in claim 1 wherein said compound is substantially a single enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, substantially an individual diastereomer, or a mixture of about 90% or more by weight of an individual diastereomer and about 10% or less by weight of any other diastereomer.

3. The compound as recited in claim 1, wherein at least one of R₁, R₂, R₃, R₄, R₅, R₆, R₇, R₈, R₉, R₁₀, R₁₁, R₁₂, R₁₃, and R₁₄ independently has deuterium enrichment of no less than about 1%.

4. The compound as recited in claim 1, wherein at least one of R₁, R₂, R₃, R₄, R₅, R₆, R₇, R₈, R₉, R₁₀, R₁₁, R₁₂, R₁₃, and R₁₄ independently has deuterium enrichment of no less than about 5%.

5. The compound as recited in claim 1, wherein at least one of R₁, R₂, R₃, R₄, R₅, R₆, R₇, R₈, R₉, R₁₀, R₁₁, R₁₂, R₁₃, and R₁₄ independently has deuterium enrichment of no less than about 10%.

6. The compound as recited in claim 1, wherein at least one of R₁, R₂, R₃, R₄, R₅, R₆, R₇, R₈, R₉, R₁₀, R₁₁, R₁₂, R₁₃, and R₁₄ independently has deuterium enrichment of no less than about 20%.

7. The compound as recited in claim 1, wherein at least one of R₁, R₂, R₃, R₄, R₅, R₆, R₇, R₈, R₉, R₁₀, R₁₁, R₁₂, R₁₃, and R₁₄ independently has deuterium enrichment of no less than about 50%.

8. The compound as recited in claim 1, wherein at least one of R₁, R₂, R₃, R₄, R₅, R₆, R₇, R₈, R₉, R₁₀, R₁₁, R₁₂, R₁₃, and R₁₄ independently has deuterium enrichment of no less than about 90%.

9. The compound as recited in claim 1, wherein at least one of R₁, R₂, R₃, R₄, R₅, R₆, R₇, R₈, R₉, R₁₀, R₁₁, R₁₂, R₁₃, and R₁₄ independently has deuterium enrichment of no less than about 98%.

10. The compound as recited in claim 1, having a structural formula selected from the group consisting of: 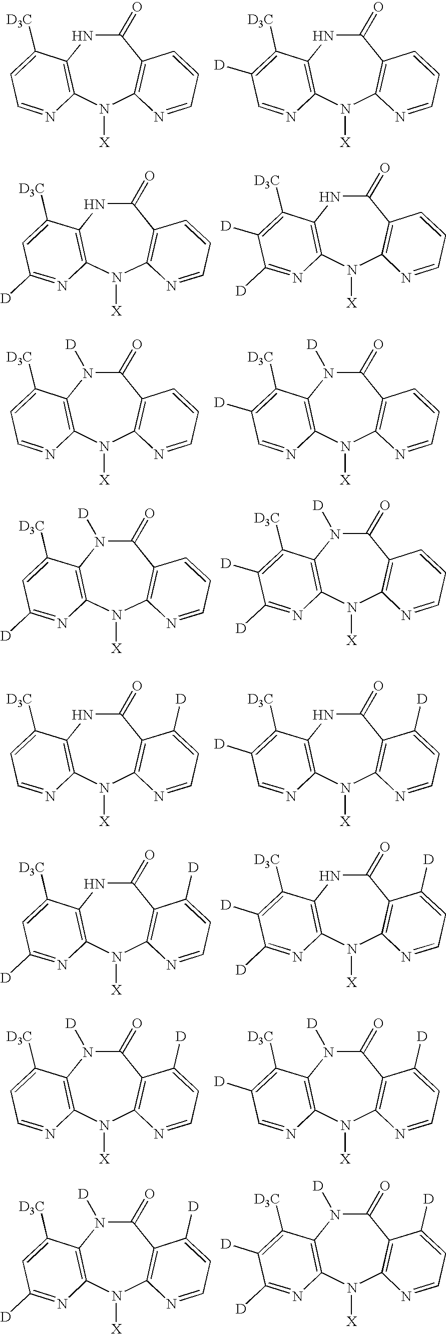 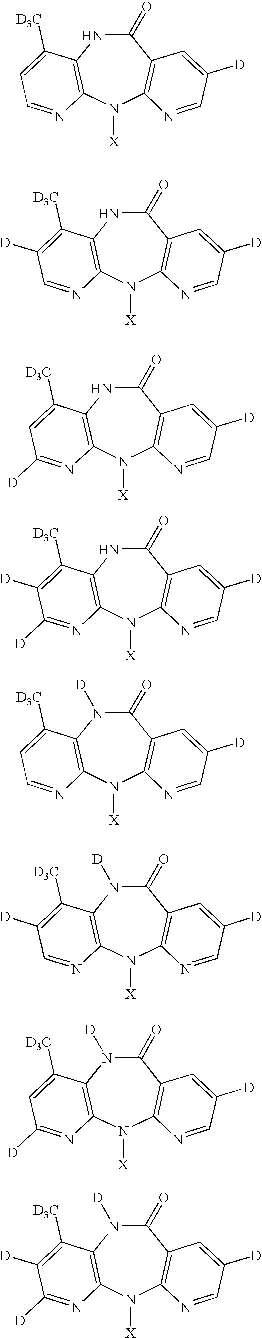 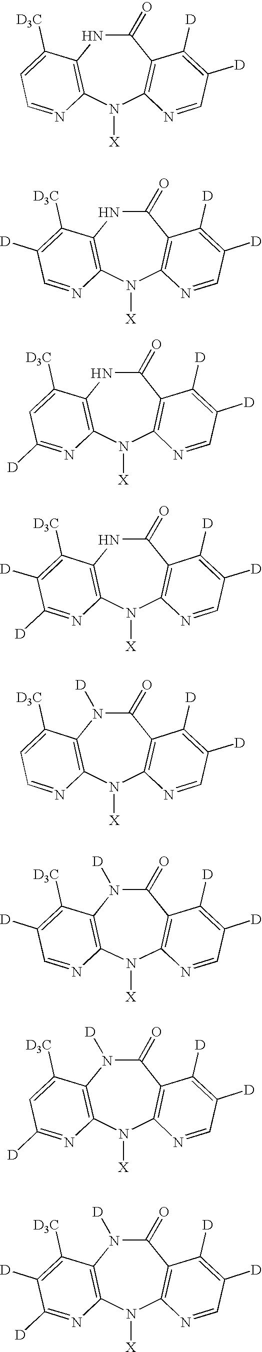 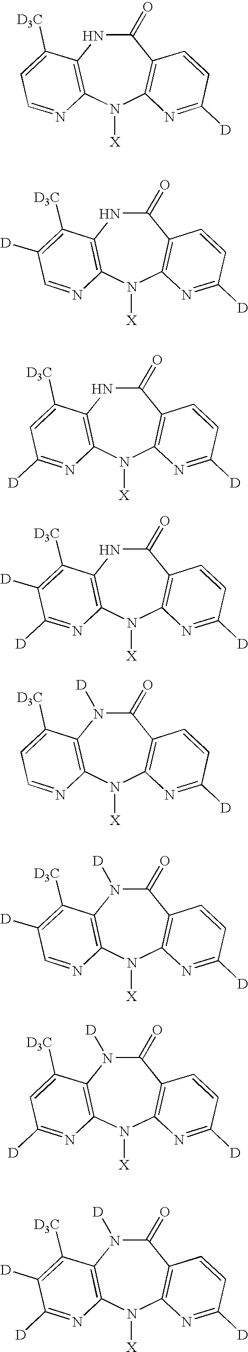 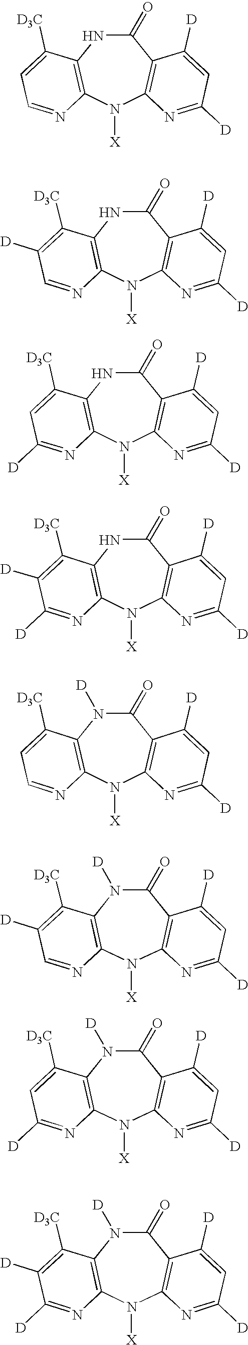 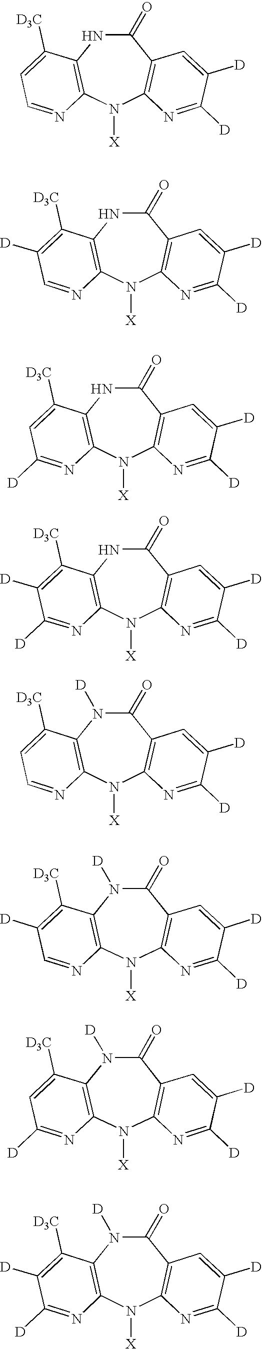 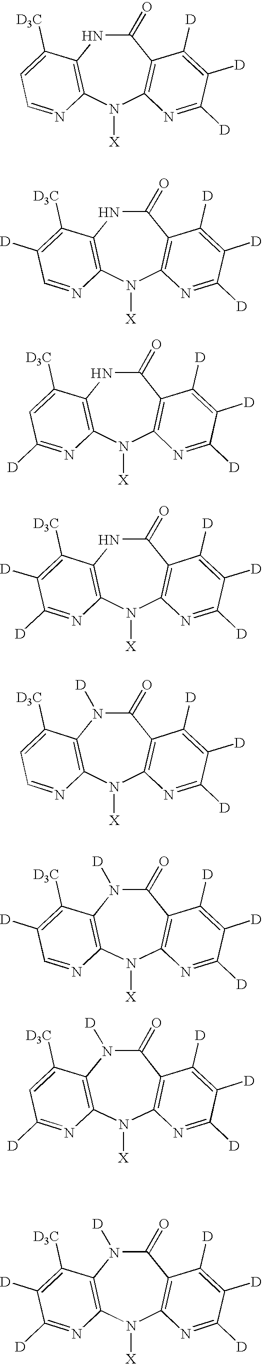
or a pharmaceutically acceptable salt, solvate, or prodrug thereof, wherein:
X is selected from the group consisting of cyclopropyl, di-cyclopropyl, d₂-cyclopropyl, d₃-cyclopropyl, d₄-cyclopropyl, and d₅-cyclopropyl, and
with the proviso that the compound cannot be

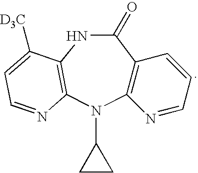

11. The compound as recited in claim 10 wherein said compound is substantially a single enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, substantially an individual diastereomer, or a mixture of about 90% or more by weight of an individual diastereomer and about 10% or less by weight of any other diastereomer.

12. The compound as recited in claim 10, wherein each of said positions represented as D have deuterium enrichment of at least 1%.

13. The compound as recited in claim 10, wherein each of said positions represented as D have deuterium enrichment of at least 5%.

14. The compound as recited in claim 10, wherein each of said positions represented as D have deuterium enrichment of at least 10%.

15. The compound as recited in claim 10, wherein each of said positions represented as D have deuterium enrichment of at least 20%.

16. The compound as recited in claim 10, wherein each of said positions represented as D have deuterium enrichment of at least 50%.

17. The compound as recited in claim 10, wherein each of said positions represented as D have deuterium enrichment of at least 90%.

18. The compound as recited in claim 10, wherein each of said positions represented as D have deuterium enrichment of at least 98%.

19. The compound as recited in claim 1, having a structural formula selected from the group consisting of: 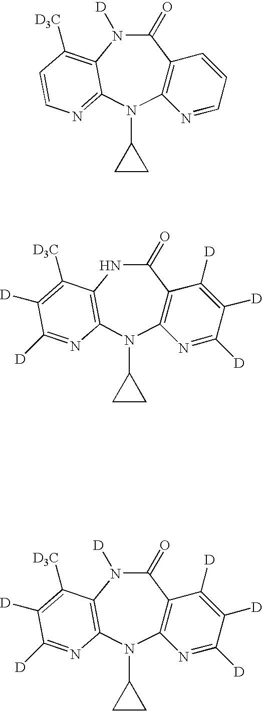
or a pharmaceutically acceptable salt, solvate, or prodrug thereof.

20. The compound as recited in claim 19 wherein said compound is substantially a single enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, substantially an individual diastereomer, or a mixture of about 90% or more by weight of an individual diastereomer and about 10% or less by weight of any other diastereomer.

21. The compound as recited in claim 19, wherein each of said positions represented as D have deuterium enrichment of at least 1%.

22. The compound as recited in claim 19, wherein each of said positions represented as D have deuterium enrichment of at least 5%.

23. The compound as recited in claim 19, wherein each of said positions represented as D have deuterium enrichment of at least 10%.

24. The compound as recited in claim 19, wherein each of said positions represented as D have deuterium enrichment of at least 20%.

25. The compound as recited in claim 19, wherein each of said positions represented as D have deuterium enrichment of at least 50%.

26. The compound as recited in claim 19, wherein each of said positions represented as D have deuterium enrichment of at least 90%.

27. The compound as recited in claim 19, wherein each of said positions represented as D have deuterium enrichment of at least 98%.

28. The compound as recited in claim 1, having a structural formula: 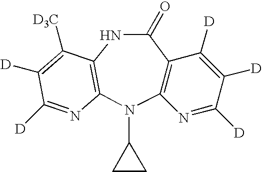
or a pharmaceutically acceptable salt, solvate, or prodrug thereof.

29. The compound as recited in claim 28 wherein said compound is substantially a single enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, substantially an individual diastereomer, or a mixture of about 90% or more by weight of an individual diastereomer and about 10% or less by weight of any other diastereomer.

30. The compound as recited in claim 28, wherein each of said positions represented as D have deuterium enrichment of at least 1%.

31. The compound as recited in claim 28, wherein each of said positions represented as D have deuterium enrichment of at least 5%.

32. The compound as recited in claim 28, wherein each of said positions represented as D have deuterium enrichment of at least 10%.

33. The compound as recited in claim 28, wherein each of said positions represented as D have deuterium enrichment of at least 20%.

34. The compound as recited in claim 28, wherein each of said positions represented as D have deuterium enrichment of at least 50%.

35. The compound as recited in claim 28, wherein each of said positions represented as D have deuterium enrichment of at least 90%.

36. The compound as recited in claim 28, wherein each of said positions represented as D have deuterium enrichment of at least 98%.

37. A pharmaceutical composition comprising a pharmaceutically acceptable carrier together with the compound as recited in claim 1.

38. The pharmaceutical composition as recited in claim 37, wherein said composition is suitable for oral, parenteral, or intravenous infusion administration.

39. The pharmaceutical composition as recited in claim 38, wherein said composition comprises a tablet or capsule.

40. The pharmaceutical composition as recited in claim 38, wherein said composition comprises a suspension.

41. The pharmaceutical composition as recited in claim 39, wherein said composition is administered in a dose of 0.5 milligrams to 1000 milligrams.

42. A pharmaceutical composition as recited in claim 37, further comprising another therapeutic agent.

43. The pharmaceutical composition as recited in claim 42, wherein the therapeutic agent is selected from the group consisting of: NRTIs, NNRTIs, protease inhibitors, entry or fusin inhibitors, integrase inhibitors, maturation inhibitors, antiviral associated agents, HIV fixed drug combinations, antifungal agents, antibacterials, antimycobacterial agents, sepsis treatments, steroidal drugs, anticoagulants, thrombolytics, non-steroidal anti-inflammatory agents, antiplatelet agents, endothelin converting enzyme (ECE) inhibitors, thromboxane receptor antagonists, potassium channel openers, thrombin inhibitors, growth factor inhibitors, platelet activating factor (PAF) antagonists, anti-platelet agents, Factor VIIa Inhibitors, Factor Xa Inhibitors, renin inhibitors, neutral endopeptidase (NEP) inhibitors, vasopepsidase inhibitors, HMG CoA reductase inhibitors, squalene synthetase inhibitors, fibrates, bile acid sequestrants, anti-atherosclerotic agents, MTP Inhibitors, calcium channel blockers, potassium channel activators, alpha-adrenergic agents, beta-adrenergic agents, antiarrhythmic agents, diuretics, anti-diabetic agents, PPAR-gamma agonists, mineralocorticoid receptor antagonists, aP2 inhibitors, phosphodiesterase inhibitors, protein tyrosine kinase inhibitors, antiinflammatories, antiproliferatives, chemotherapeutic agents, immunosuppressants, anticancer agents, cytotoxic agents, antimetabolites, farnesyl-protein transferase inhibitors, hormonal agents, microtubule-disruptor agents, microtubule-stablizing agents, topoisomerase inhibitors, prenyl-protein transferase inhibitors, cyclosporins, TNF-alpha inhibitors, cyclooxygenase-2 (COX-2) inhibitors, gold compounds, and platinum coordination complexes.

44. The pharmaceutical composition as recited in claim 43, wherein the therapeutic agent is a NRTI.

45. The pharmaceutical composition as recited in claim 44, wherein the NRTI is selected from the group consisting of abacavir, didanosine, emtricitabine, lamivudine, stavudine, zidovudine, tenofovir, apricitabine, stampidine, elvucitabine, racivir and zalcitabine.

46. The pharmaceutical composition as recited in claim 45, wherein the NRTI is zidovudine.

47. The pharmaceutical composition as recited in claim 46, further comprising lamivudine.

48. The pharmaceutical composition as recited in claim 45, wherein the NRTI is tenofovir.

49. The pharmaceutical composition as recited in claim 48, further comprising emtricitabine.

50. The pharmaceutical composition as recited in claim 45, wherein the NRTI is stavudine.

51. The pharmaceutical composition as recited in claim 50, further comprising lamivudine.

52. The pharmaceutical composition as recited in claim 44, further comprising another NRTI.

53. The pharmaceutical composition as recited in claim 43, wherein the therapeutic agent is a NNRTI.

54. The pharmaceutical composition as recited in claim 53, wherein the NNRTI is selected from the group consisting of avirenz, nevirapine, etravirine, rilpivirine, loviride and delavirdine.

55. The pharmaceutical composition as recited in claim 43, wherein the therapeutic agent is a protease inhibitor.

56. The pharmaceutical composition as recited in claim 55, wherein the protease inhibitor is selected from the group consisting of atazanavir, darunavir, fosamprenavir, lopinavir, nelfinavir, ritonavir, saquinavir, tipranavir, amprenavir and indinavir.

57. The pharmaceutical composition as recited in claim 43, wherein the therapeutic agent is a entry or fusion inhibitor.

58. The pharmaceutical composition as recited in claim 57, wherein the entry or fusion inhibitor is selected from the group consisting of enfuvirtide, maraviroc, PRO140 and vicriviroc.

59. The pharmaceutical composition as recited in claim 43, wherein the therapeutic agent is a integrase inhibitor.

60. The pharmaceutical composition as recited in claim 59, wherein the integrase inhibitor is selected from the group consisting of raltegravir, and elvitegravir.

61. The pharmaceutical composition as recited in claim 43, wherein the therapeutic agent is a maturation inhibitor.

62. The pharmaceutical composition as recited in claim 61, wherein the maturation inhibitor is selected from the group consisting of bevirimat and vivecon.

63. The pharmaceutical composition as recited in claim 43, wherein the therapeutic agent is an antiviral associated agent.

64. The pharmaceutical composition as recited in claim 63, wherein the antiviral associated agent is selected from the group consisting of foscarnet, chloroquine, quinoline, grapefruit juice, hydroxyurea, leflunomide, mycophenolic acid, resveratrol, ritonavir, epigallocatechin gallate, portmanteau inhibitors, Globoidnan A, griffithsin, diarylpyrimidines, and Calanolide A.

65. The pharmaceutical composition as recited in claim 43, wherein the therapeutic agent is an HIV fixed drug combination.

66. The pharmaceutical composition as recited in claim 65, wherein the HIV fixed drug combination is selected from the group consisting of Combivir®, Atripla®, Trizivir®, Truvada®, Kaletra®, and Epzicom®.

67. A pharmaceutical composition comprising a pharmaceutically acceptable carrier together with the compound as recited in claim 19.

68. The pharmaceutical composition as recited in claim 67, wherein said composition is suitable for oral, parenteral, or intravenous infusion administration.

69. The pharmaceutical composition as recited in claim 68, wherein said composition comprises a tablet or capsule.

70. The pharmaceutical composition as recited in claim 68, wherein said composition comprises a suspension.

71. The pharmaceutical composition as recited in claim 69, wherein said composition is administered in a dose of 0.5 milligrams to 1000 milligrams.

72. A pharmaceutical composition as recited in claim 67, further comprising another therapeutic agent.

73. The pharmaceutical composition as recited in claim 72, wherein the therapeutic agent is selected from the group consisting of: NRTIs, NNRTIs, protease inhibitors, entry or fusin inhibitors, integrase inhibitors, maturation inhibitors, antiviral associated agents, HIV fixed drug combinations, antifungal agents, antibacterials, antimycobacterial agents, sepsis treatments, steroidal drugs, anticoagulants, thrombolytics, non-steroidal anti-inflammatory agents, antiplatelet agents, endothelin converting enzyme (ECE) inhibitors, thromboxane receptor antagonists, potassium channel openers, thrombin inhibitors, growth factor inhibitors, platelet activating factor (PAF) antagonists, anti-platelet agents, Factor VIIa Inhibitors, Factor Xa Inhibitors, renin inhibitors, neutral endopeptidase (NEP) inhibitors, vasopepsidase inhibitors, HMG CoA reductase inhibitors, squalene synthetase inhibitors, fibrates, bile acid sequestrants, anti-atherosclerotic agents, MTP Inhibitors, calcium channel blockers, potassium channel activators, alpha-adrenergic agents, beta-adrenergic agents, antiarrhythmic agents, diuretics, anti-diabetic agents, PPAR-gamma agonists, mineralocorticoid receptor antagonists, aP2 inhibitors, phosphodiesterase inhibitors, protein tyrosine kinase inhibitors, antiinflammatories, antiproliferatives, chemotherapeutic agents, immunosuppressants, anticancer agents, cytotoxic agents, antimetabolites, farnesyl-protein transferase inhibitors, hormonal agents, microtubule-disruptor agents, microtubule-stablizing agents, topoisomerase inhibitors, prenyl-protein transferase inhibitors, cyclosporins, TNF-alpha inhibitors, cyclooxygenase-2 (COX-2) inhibitors, gold compounds, and platinum coordination complexes.

74. The pharmaceutical composition as recited in claim 73, wherein the therapeutic agent is a NRTI.

75. The pharmaceutical composition as recited in claim 74, wherein the NRTI is selected from the group consisting of abacavir, didanosine, emtricitabine, lamivudine, stavudine, zidovudine, tenofovir, apricitabine, stampidine, elvucitabine, racivir and zalcitabine.

76. The pharmaceutical composition as recited in claim 75, wherein the NRTI is zidovudine.

77. The pharmaceutical composition as recited in claim 76, further comprising lamivudine.

78. The pharmaceutical composition as recited in claim 75, wherein the NRTI is tenofovir.

79. The pharmaceutical composition as recited in claim 78, further comprising emtricitabine.

80. The pharmaceutical composition as recited in claim 75, wherein the NRTI is stavudine.

81. The pharmaceutical composition as recited in claim 80, further comprising lamivudine.

82. The pharmaceutical composition as recited in claim 74, further comprising another NRTI.

83. The pharmaceutical composition as recited in claim 73, wherein the therapeutic agent is a NNRTI.

84. The pharmaceutical composition as recited in claim 83, wherein the NNRTI is selected from the group consisting of avirenz, nevirapine, etravirine, rilpivirine, loviride and delavirdine.

85. The pharmaceutical composition as recited in claim 73, wherein the therapeutic agent is a protease inhibitor.

86. The pharmaceutical composition as recited in claim 85, wherein the protease inhibitor is selected from the group consisting of atazanavir, darunavir, fosamprenavir, lopinavir, nelfinavir, ritonavir, saquinavir, tipranavir, amprenavir and indinavir.

87. The pharmaceutical composition as recited in claim 73, wherein the therapeutic agent is a entry or fusion inhibitor.

88. The pharmaceutical composition as recited in claim 87, wherein the entry or fusion inhibitor is selected from the group consisting of enfuvirtide, maraviroc, PRO140 and vicriviroc.

89. The pharmaceutical composition as recited in claim 73, wherein the therapeutic agent is a integrase inhibitor.

90. The pharmaceutical composition as recited in claim 89, wherein the integrase inhibitor is selected from the group consisting of raltegravir, and elvitegravir.

91. The pharmaceutical composition as recited in claim 73, wherein the therapeutic agent is a maturation inhibitor.

92. The pharmaceutical composition as recited in claim 91, wherein the maturation inhibitor is selected from the group consisting of bevirimat and vivecon.

93. The pharmaceutical composition as recited in claim 73, wherein the therapeutic agent is an antiviral associated agent.

94. The pharmaceutical composition as recited in claim 93, wherein the antiviral associated agent is selected from the group consisting of foscarnet, chloroquine, quinoline, grapefruit juice, hydroxyurea, leflunomide, mycophenolic acid, resveratrol, ritonavir, epigallocatechin gallate, portmanteau inhibitors, Globoidnan A, griffithsin, diarylpyrimidines, and Calanolide A.

95. The pharmaceutical composition as recited in claim 73, wherein the therapeutic agent is an HIV fixed drug combination.

96. The pharmaceutical composition as recited in claim 95, wherein the HIV fixed drug combination is selected from the group consisting of Combivir®, Atripla®, Trizivir®, Truvada®, Kaletra®, and Epzicom®.

97. A method of treating a subject suffering from an infectious disorder, comprising administering to said subject a therapeutically effective amount of a compound having structural Formula (II) 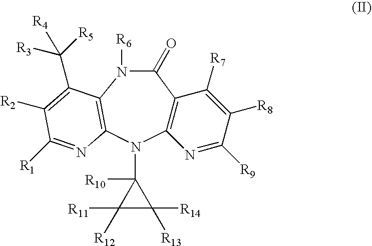
or a pharmaceutically acceptable salt, solvate, or prodrug thereof, wherein:
R₁, R₂, R₃, R₄, R₅, R₆, R₇, R₈, R₉, R₁₀, R₁₁, R₁₂, R₁₃, and R₁₄ are independently selected from the group consisting of hydrogen and deuterium; and
at least one of R₁, R₂, R₃, R₄, R₅, R₆, R₇, R₈, R₉, R₁₀, R₁₁, R₁₂, R₁₃, and R₁₄ is deuterium.

98. The method as recited in claim 97, wherein said infectious disorder is caused by a virus.

99. The method as recited in claim 98, wherein said virus is a retrovirus.

100. The method as recited in claim 99, wherein said retrovirus is selected from the group consisting of alpharetrovirus, betaretrovirus, gammaretrovirus, deltaretrovirus, epsilonretrovirus, lentivirus, spumavirus, and endogenous retrovirus.

101. The method as recited in claim 100, wherein said retrovirus is lentivirus.

102. The method as recited in claim 101, wherein said lentivirus is HIV type 1.

103. The method as recited in claim 97, wherein said compound has at least one of the following properties:
a) decreased inter-individual variation in plasma levels of said compound or a metabolite thereof as compared to the non-isotopically enriched compound;
b) increased average plasma levels of said compound per dosage unit thereof as compared to the non-isotopically enriched compound;
c) decreased average plasma levels of at least one metabolite of said compound per dosage unit thereof as compared to the non-isotopically enriched compound;
d) increased average plasma levels of at least one metabolite of said compound per dosage unit thereof as compared to the non-isotopically enriched compound; and
e) an improved clinical effect during the treatment in said subject per dosage unit thereof as compared to the non-isotopically enriched compound.

104. The method as recited in claim 97, wherein said compound has at least two of the following properties:
a) decreased inter-individual variation in plasma levels of said compound or a metabolite thereof as compared to the non-isotopically enriched compound;
b) increased average plasma levels of said compound per dosage unit thereof as compared to the non-isotopically enriched compound;
c) decreased average plasma levels of at least one metabolite of said compound per dosage unit thereof as compared to the non-isotopically enriched compound;
d) increased average plasma levels of at least one metabolite of said compound per dosage unit thereof as compared to the non-isotopically enriched compound; and
e) an improved clinical effect during the treatment in said subject per dosage unit thereof as compared to the non-isotopically enriched compound.

105. The method as recited in claim 97, wherein said compound has a decreased metabolism by at least one polymorphically-expressed cytochrome P₄₅₀ isoform in said subject per dosage unit thereof as compared to the non-isotopically enriched compound.

106. The method as recited in claim 105, wherein said cytochrome P₄₅₀ isoform is selected from the group consisting of CYP2C8, CYP2C9, CYP2C19, and CYP2D6.

107. The method as recited in claim 97, wherein said compound is characterized by decreased inhibition of at least one cytochrome P₄₅₀ or monoamine oxidase isoform in said subject per dosage unit thereof as compared to the non-isotopically enriched compound.

108. The method as recited in claim 107, wherein said cytochrome P₄₅₀ or monoamine oxidase isoform is selected from the group consisting of CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4×1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, CYP51, MAO_{A}, and MAO_{B}.

109. The method as recited in claim 97, wherein the method affects the treatment of the disorder while reducing or eliminating a deleterious change in a diagnostic hepatobiliary function endpoint, as compared to the corresponding non-isotopically enriched compound.

110. The method as recited in claim 109, wherein the diagnostic hepatobiliary function endpoint is selected from the group consisting of alanine aminotransferase (“ALT”), serum glutamic-pyruvic transaminase (“SGPT”), aspartate aminotransferase (“AST,” “SGOT”), ALT/AST ratios, serum aldolase, alkaline phosphatase (“ALP”), ammonia levels, bilirubin, gamma-glutamyl transpeptidase (“GGTP,” “γ-GTP,” “GGT”), leucine aminopeptidase (“LAP”), liver biopsy, liver ultrasonography, liver nuclear scan, 5′-nucleotidase, and blood protein.

111. A method of treating a subject suffering from an infectious disorder, comprising administering to said subject a therapeutically effective amount of a compound selected from the group consisting of: 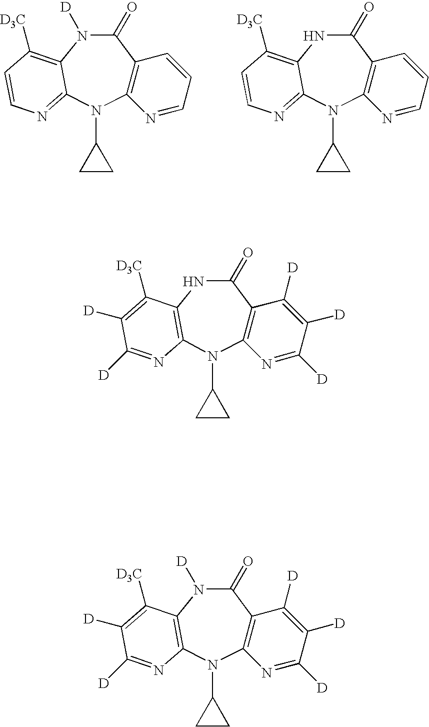
or a pharmaceutically acceptable salt, solvate, or prodrug thereof.

112. The method as recited in claim 111, wherein said infectious disorder is caused by a virus.

113. The method as recited in claim 112, wherein said virus is a retrovirus.

114. The method as recited in claim 113, wherein said retrovirus is selected from the group consisting of alpharetrovirus, betaretrovirus, gammaretrovirus, deltaretrovirus, epsilonretrovirus, lentivirus, spumavirus, and endogenous retrovirus.

115. The method as recited in claim 114, wherein said retrovirus is lentivirus.

116. The method as recited in claim 115, wherein said lentivirus is HIV type 1.

117. The method as recited in claim 111, wherein said compound has at least one of the following properties:
a) decreased inter-individual variation in plasma levels of said compound or a metabolite thereof as compared to the non-isotopically enriched compound;
b) increased average plasma levels of said compound per dosage unit thereof as compared to the non-isotopically enriched compound;
c) decreased average plasma levels of at least one metabolite of said compound per dosage unit thereof as compared to the non-isotopically enriched compound;
d) increased average plasma levels of at least one metabolite of said compound per dosage unit thereof as compared to the non-isotopically enriched compound; and
e) an improved clinical effect during the treatment in said subject per dosage unit thereof as compared to the non-isotopically enriched compound.

118. The method as recited in claim 111, wherein said compound has at least two of the following properties:
a) decreased inter-individual variation in plasma levels of said compound or a metabolite thereof as compared to the non-isotopically enriched compound;
b) increased average plasma levels of said compound per dosage unit thereof as compared to the non-isotopically enriched compound;
c) decreased average plasma levels of at least one metabolite of said compound per dosage unit thereof as compared to the non-isotopically enriched compound;
d) increased average plasma levels of at least one metabolite of said compound per dosage unit thereof as compared to the non-isotopically enriched compound; and
e) an improved clinical effect during the treatment in said subject per dosage unit thereof as compared to the non-isotopically enriched compound.

119. The method as recited in claim 111, wherein said compound has a decreased metabolism by at least one polymorphically-expressed cytochrome P₄₅₀ isoform in said subject per dosage unit thereof as compared to the non-isotopically enriched compound.

120. The method as recited in claim 119, wherein said cytochrome P₄₅₀ isoform is selected from the group consisting of CYP2C8, CYP2C9, CYP2C19, and CYP2D6.

121. The method as recited in claim 111, wherein said compound is characterized by decreased inhibition of at least one cytochrome P₄₅₀ or monoamine oxidase isoform in said subject per dosage unit thereof as compared to the non-isotopically enriched compound.

122. The method as recited in claim 121, wherein said cytochrome P₄₅₀ or monoamine oxidase isoform is selected from the group consisting of CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4×1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, CYP51, MAO_{A}, and MAO_{B}.

123. The method as recited in claim 111, wherein the method affects the treatment of the disorder while reducing or eliminating a deleterious change in a diagnostic hepatobiliary function endpoint, as compared to the corresponding non-isotopically enriched compound.

124. The method as recited in claim 123, wherein the diagnostic hepatobiliary function endpoint is selected from the group consisting of alanine aminotransferase (“ALT”), serum glutamic-pyruvic transaminase (“SGPT”), aspartate aminotransferase (“AST,” “SGOT”), ALT/AST ratios, serum aldolase, alkaline phosphatase (“ALP”), ammonia levels, bilirubin, gamma-glutamyl transpeptidase (“GGTP,” “γ-GTP,” “GGT”), leucine aminopeptidase (“LAP”), liver biopsy, liver ultrasonography, liver nuclear scan, 5′-nucleotidase, and blood protein.
